(19) United States
(12) Patent Application Publication
(10) Pub. No.: US 2020/0163938 A1
(43) Pub. Date: May 28, 2020

(54) PHARMACEUTICAL COMPOSITION COMPRISING IRON CHELATOR EXHIBITING ANTITUMOR ACTIVITY, ANTIBACTERIAL ACTIVITY AND/OR ANTIVIRUS ACTIVITY, AND HAVING REDUCED SIDE EFFECTS

(71) Applicant: Disease Adsorption System Technologies Co., Ltd., Kanazawa-shi (JP)

(72) Inventors: Yuzo NISHIDA, Ishikawa (JP); Toshiaki OHARA, Okayama (JP); Kazuhiro OMORI, Okayama (JP); Yasuko TOMONO, Okayama (JP)

(73) Assignee: Disease Adsorption System Technologies Co., Ltd., Kanazawa-shi (JP)

(21) Appl. No.: 16/611,677

(22) PCT Filed: May 10, 2018
(86) PCT No.: PCT/JP2018/018032

§ 371 Date: Nov. 7, 2019

(30) Foreign Application Priority Data
May 11, 2017 (JP) 2017-094954
Jan. 25, 2018 (JP) 2018-010757

(51) Int. Cl. A61K 31/4172; A61K 47/36; A61P 31/04; A61K 47/26

(52) U.S. Cl. CPC A61K 31/4172; A61K 47/26; A61P 31/04; A61K 47/36

(57) ABSTRACT

The present invention provides a pharmaceutical composition comprising an iron chelating agent exhibiting antitumor activity, antimicrobial activity, and/or antivirus activity and having reduced side effects. Specifically, the present invention provides a pharmaceutical composition for use in treatment of cancer or infectious disease, comprising an iron chelating agent that selectively binds to biologically unstable iron, rather than to transferrin-bound iron.

### CROSS-REFERENCES TO RELATED APPLICATIONS

This application claims priority from Japanese Patent Application No. 2017-094954 (filing date: May 11, 2017) and Japanese Patent Application No. 2018-010757 (filing date: Jan. 25, 2018) and the disclosures of which are incorporated herein by reference in their entirety.

### TECHNICAL FIELD

The present invention relates to a pharmaceutical composition comprising an iron chelating agent exhibiting antitumor activity, antimicrobial activity, and/or antivirus activity and having reduced side effects.

### BACKGROUND ART

Battle with cancer is also referred to as battle with anticancer agents, such that chemotherapy for cancer is problematic in significant side effects. Iron chelating agents have been developed for treating cancer, since it is considered that lowering the blood iron level can lead to improvement in the prognosis of cancer patients (Non Patent Literature 1 to 3).

Iron chelation is known to exhibit antimicrobial effects, and the use of an iron chelating agent as an antimicrobial agent is under study.

Chelating agents for selectively removing biologically unstable iron have been proposed (Patent Literature 1 and 2). Biologically unstable iron is considered to be unnecessary for living bodies. Hence, it is not considered that cancer and infectious diseases can be treated by removing such biologically unstable iron.

### CITATION LIST

### Patent Literature

Patent Literature 1: WO2012/096183
Patent Literature 2: WO2016/052488

### Non Patent Literature

Non Patent Literature 1: Lee S. et al., J Cancer., 2016; 12, 7(8):957-964
Non Patent Literature 2: Tingting H. et al., Saudi Med J., 2017; 38(3):268-275
Non Patent Literature 3: Ji M. et al., Tumour Biol., 2014; 35(10): 10195-1019949 (4):1351-1359

### SUMMARY OF INVENTION

The present invention provides a pharmaceutical composition comprising an iron chelating agent exhibiting antitumor activity, antimicrobial activity, and/or antivirus activity and having reduced side effects.

The present inventors have focused on the fact that most side effects of existing iron chelating agents are caused through administration, and the safety for patients with cancer has not yet been established, and thus have developed a novel chelating agent having safety higher than that of existing iron chelating agents. Specifically, the present inventors have revealed that an iron chelating agent selectively binding to biologically unstable iron rather than binding to transferrin-bound iron has antitumor activity and antimicrobial activity, and thus have completed the present invention.

Specifically, the present invention provides the following (1) to (28).

(1) A pharmaceutical composition for use in treatment of cancer, comprising an iron chelating agent, wherein

the iron chelating agent has a substrate selected from a polymer backbone, glucosamine, and histidine; and an aromatic ring bonded to the substrate through an —NH—CH₂— bond, wherein the aromatic ring has one or two first functional groups, which are each a hydroxyl group; and one or two second functional groups selected from a hydroxyl group, a carboxylic acid group, and a functional group represented by formula (I):

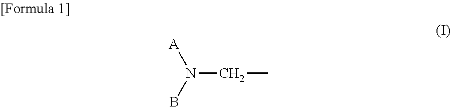

wherein A is —CH₃, —CH₂—CH₃, —CH₂—C₆H₅, —CH₂—C₅H₄N, or —CH₂—COOH; and B is —CH₂—COOH, and wherein
the second functional group is located in the ortho position relative to at least one of the first functional groups.
(2) The pharmaceutical composition according to (1) above, wherein the polymer backbone is a chitosan backbone.
(3) The pharmaceutical composition according to (1) or (2) above, wherein the aromatic ring has the following structure:

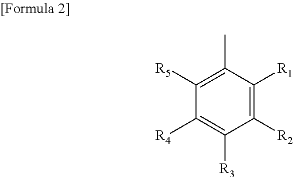

wherein any one of R₁ to R₅ is OH; the ring has at least OH or COOH in the ortho position relative to the OH; and the other groups are selected from H, OH, COOH, CH₃, and —N(CH₃)—CH₂—COOH.
(4) The pharmaceutical composition according to (1) above, wherein the substrate is glucosamine.
(5) The pharmaceutical composition according to (4) above, wherein the aromatic ring has the following structure:

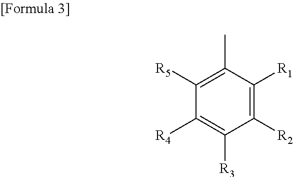

wherein any one of R₁ to R₅ is OH; the ring has at least OH or COOH in the ortho position relative to the OH; and the other groups are selected from H, OH, COOH, CH₃, and —N(CH₃)—CH₂—COOH.
(6) The pharmaceutical composition according to (5) above, wherein R₁ is H or OH; R₂ and R₃ are each OH; and R₄ and R₅ are each H.
(7) The pharmaceutical composition according to (1) above, wherein the iron chelating agent has the following structure:

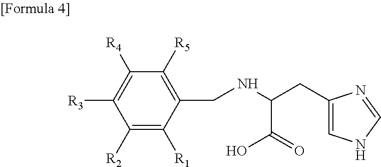

wherein any one of R₁ to R₅ is OH; the ring has at least OH or COOH in the ortho position relative to the OH; and the other groups are selected from H, OH, COOH, CH₃, and —N(CH₃)—CH₂—COOH.
(8) The pharmaceutical composition according to (7) above, wherein the chelating agent is in the form of hydrochloride salt.
(9) The pharmaceutical composition according to (7) or (8) above, wherein any one of R₁ to R₅ is OH; the ring has at least OH or COOH in the ortho position relative to the OH.
(10) The pharmaceutical composition according to (9) above, wherein

R₁ to R₃ are each OH; and R₄ and R₅ are each H; or

R₁ is H; one of R₂ and R₃ is OH; and the other is COOH; and R₄ and R₅ are each H.

(11) An antimicrobial agent comprising an iron chelating agent, wherein

the iron chelating agent has a substrate selected from a polymer backbone, glucosamine, and histidine; and an aromatic ring bonded to the substrate through an —NH—CH₂— bond, wherein the aromatic ring has one or two first functional groups, which are each a hydroxyl group; and one or two second functional groups selected from a hydroxyl group, a carboxylic acid group, and a functional group represented by formula (I):

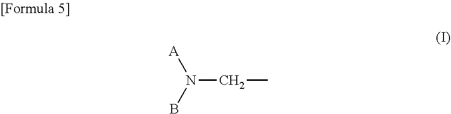

wherein A is —CH₃, —CH₂—CH₃, —CH₂—C₆H₅, —CH₂—C₅H₄N, or —CH₂—COOH; and B is —CH₂—COOH, and wherein
the second functional group is located in the ortho position relative to at least one of the first functional groups.
(12) The antimicrobial agent according to (11) above, wherein the substrate is glucosamine.
(13) The antimicrobial agent according to (11) above, wherein

the iron chelating agent has glucosamine; and an aromatic ring bonded to glucosamine through an —NH—CH₂— bond, wherein the aromatic ring has one or two first functional groups, which are each a hydroxyl group; and one or two second functional groups selected from a hydroxyl group, a carboxylic acid group, and a functional group represented by formula (I):

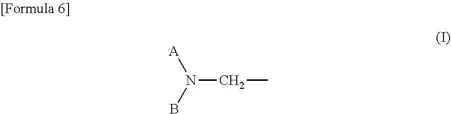

wherein A is —CH₃, —CH₂—CH₃, —CH₂—C₆H₅, —CH₂—C₅H₄N or —CH₂—COOH; and B is —CH₂—COOH, and wherein
the second functional group is located in the ortho position relative to at least one of the first functional groups.
(14) The antimicrobial agent according to (12) or (13) above, wherein the aromatic ring has the following structure:

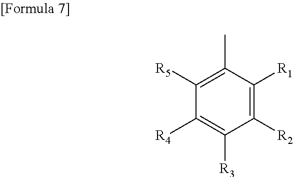

wherein any one of R₁ to R₅ is OH; the ring has at least OH or COOH in the ortho position relative to the OH; and the other groups are selected from H, OH, COOH, CH₃, and —N(CH₃)—CH₂—COOH.
(15) The antimicrobial agent according to (14) above, wherein R₁ is H or OH; R₂ and R₃ are each OH; and R₄ and R₅ are each H.
(16) The antimicrobial agent according to any one of (11) to (15) above, wherein

the antimicrobial agent is an antimicrobial agent for use against an oral bacterium selected from the group consisting of S. mutans, A. actinomycetemcomitans and P. gingivalis.

(17) The antimicrobial agent according to (15) above, wherein

the antimicrobial agent is an antimicrobial agent for use against an oral bacterium selected from the group consisting of S. mutans, A. actinomycetemcomitans and P. gingivalis.

(18) The antimicrobial agent according to (11) above, wherein

the antimicrobial agent is an antimicrobial agent for use against an oral bacterium selected from the group consisting of S. mutans and P. gingivalis, and

the iron chelating agent has chitosan as a substrate.

(19) The antimicrobial agent according to any one of (11) to (15) above, wherein

the antimicrobial agent is an antimicrobial agent for use against S. aureus or C. albicans, and

the iron chelating agent has a chitosan backbone as the polymer backbone.

(20) The antimicrobial agent according to (15) above, wherein

the antimicrobial agent is an antimicrobial agent for use against S. aureus.

(21) The antimicrobial agent according to (15) above, wherein

the antimicrobial agent is an antimicrobial agent for use against P. aeruginosa.

(22) A pharmaceutical composition for use in treatment of cancer, comprising

an iron chelating agent having selectivity for biologically unstable iron rather than for transferrin-bound iron.

(22A) The pharmaceutical composition according to (22) above, comprising

the iron chelating agent defined in (1) above.

(23) An antimicrobial agent comprising an iron chelating agent having selectivity for biologically unstable iron rather than for transferrin-bound iron.
(23A) The antimicrobial agent according to (23) above, comprising the iron chelating agent defined in (1) above.
(24) A pharmaceutical composition for use in treatment of viral infection comprising an iron chelating agent, wherein

the iron chelating agent has a substrate selected from a polymer backbone, glucosamine, and histidine; and an aromatic ring, wherein the aromatic ring has one or two first functional groups, which are each a hydroxyl group; and one or two second functional groups selected from a hydroxyl group, a carboxylic acid group, and a functional group represented by formula (I):

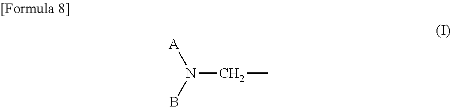

wherein A is —CH₃, —CH₂—CH₃, —CH₂—C₆H₅, —CH₂—C₅H₄N, or —CH₂—COOH; and B is —CH₂—COOH, and wherein
the second functional group is located in the ortho position relative to at least one of the first functional groups.
(25) The pharmaceutical composition according to (24) above, wherein the aromatic ring has the following structure:

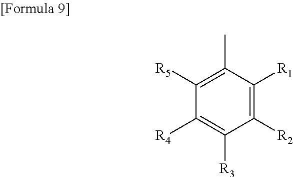

wherein any one of R₁ to R₅ is OH; the ring has at least OH or COOH in the ortho position relative to the OH; and the other groups are selected from H, OH, COOH, CH₃, and —N(CH₃)—CH₂—COOH.
(26) The pharmaceutical composition according to (25) above, wherein R₁ is H or OH; R₂ and R₃ are each OH; and R₄ and R₅ are each H.
(27) The pharmaceutical composition according to (25) above, wherein R₁ and R₅ are each H; and R₂ to R₄ are each OH.
(28) A pharmaceutical composition for use in treatment of viral infection, comprising an iron chelating agent having selectivity for biologically unstable iron rather than for transferrin-bound iron.

### BRIEF DESCRIPTION OF DRAWINGS

FIG. 1A depicts antitumor effects exhibited by the chelating agents of the present invention. A549 cells were used and the cell viability was confirmed using trypan blue.

FIG. 1B depicts antitumor effects exhibited by the chelating agents of the present invention. A549 cells were used and the cell viability was confirmed by an XTT method.

FIG. 2 depicts antitumor effects exhibited by the chelating agents of the present invention. PLC cells were used and the cell viability was confirmed using trypan blue.

FIG. 3 depicts antitumor effects exhibited by the chelating agents of the present invention. HCT116 cells were used and the cell viability was confirmed by the XTT method.

FIG. 4A depicts that the chelating agent of the present invention exhibited in vivo antitumor effects.

FIG. 4B depicts body weight changes after administration of the chelating agent of the present invention.

FIG. 5A depicts body weight changes after administration of the chelating agent of the present invention (test group 6).

FIG. 5B depicts body weight changes after administration of the chelating agent of the present invention (test group 9).

FIG. 5C depicts body weight changes after administration of the chelating agent of the present invention (test group 10).

FIG. 6A depicts that the chelating agents of the present invention exhibited antimicrobial effects (effects of decreasing the cell count) on S. mutans.

FIG. 6B depicts that the chelating agents of the present invention exhibited antimicrobial effects (effects of inhibiting ATP activity) on S. mutans.

FIG. 7A depicts that the chelating agents of the present invention exhibited antimicrobial effects (effects of decreasing the cell count) on A. actinomycetemcomitans.

FIG. 7B depicts that the chelating agents of the present invention exhibited antimicrobial effects (effects of inhibiting ATP activity) on A. actinomycetemcomitans.

FIG. 8 depicts that the chelating agents of the present invention exhibited antimicrobial effects on P. gingivalis.

FIG. 9 depicts the antitumor effects of the chelating agents of the present invention. MCF-7 cells were used and the cell viability was confirmed by the XTT method.

FIG. 10 depicts the antitumor effects of the chelating agents of the present invention. HSC-2 cells were used and the cell viability was confirmed by the XTT method.

FIG. 11A depicts the apoptosis-inducing effects of the chelating agents of the present invention on tumor cells. HSC-2 cells were used and stained by a TUNEL method.

FIG. 11B depicts the apoptosis-inducing effects of the chelating agents of the present invention on tumor cells. HCT116 cells were used and stained by the TUNEL method.

FIG. 11C depicts the apoptosis-inducing effects of the chelating agents of the present invention on tumor cells. A549 cells were used and stained by the TUNEL method.

FIG. 12 depicts the apoptosis-inducing effects of the chelating agents of the present invention on tumor cells. Western blot was carried out using HSC-2, MCF-7 and A549 cells, and the cell lysates were analyzed using antibodies against factors indicated in FIG. 12.

FIG. 13A depicts that the chelating agent of the present invention exhibited in vivo antitumor effects, and the effect of decreasing body weight was not confirmed at doses where the agent exhibited antitumor effects.

FIG. 13B depicts that the chelating agent of the present invention had in vivo apoptosis-inducing effects on tumor cells.

FIG. 14 depicts that the chelating agent of the present invention had in vivo apoptosis-inducing effects on tumor cells.

FIG. 15A depicts that the chelating agent of the present invention was capable of inhibiting viral infection.

FIG. 15B depicts that Desferal capable of chelating both transferrin-bound iron and biologically unstable iron was unable to inhibit viral infection.

### SPECIFIC DESCRIPTION OF INVENTION

In the Description, the term “subject” refers to mammals, and can be particularly the primate including humans, dogs, cats, cow, pigs, goats, or sheep.

In the Description, the term “treatment” is used in the sense of both therapy (therapeutic treatment) and prophylaxis (prophylactic treatment). In the Description, the term “therapy” refers to treatment, recovery (cure), prevention or improved amelioration of diseases or disorders, or reduction of the rate of disease or disorder progression. In the Description, the term “prophylaxis” refers to lowering the possibility of the onset of diseases or pathological conditions, or, delaying the onset of diseases or pathological conditions.

In the Description, the term “antimicrobial effects” is used in the sense of an effect of suppressing microbial growth, an effect of decreasing the rate of microbial growth, an effect of stopping microbial growth, an effect of reducing microbial count, and an effect of killing off microbes. In the Description, the term “antimicrobial agent” refers to a composition to be used for antimicrobial applications. In the Description, such an antimicrobial agent can be a pharmaceutical composition when administered to a subject.

In the Description, the term “antiviral effect” is used in the sense of an effect of suppressing viral growth, an effect of decreasing the viral growth rate, an effect of stopping viral growth, an effect of decreasing viral count, and an effect of suppressing viral cytotoxicity.

In the Description, the terms “therapeutically effective amount” refers to an amount of a drug, which is effective for treating (prophylactic or therapeutic treatment” diseases or conditions. The therapeutically effective amount of a drug can decrease the symptom worsening rate of a disease or conditions, stop the worsening of the symptoms, improve the symptoms, cure the symptoms, or suppress the onset or development of the symptoms.

In the Description, the term “iron chelating agent” is used in the sense of a chelating agent capable of chelating iron from both transferrin-bound iron and biologically unstable iron. In the Description, the term “polymeric iron chelating agent” refers to a chelating agent having an iron chelating site linked to the polymer backbone (e.g., polymer). In the Description, the term “iron chelating agent” includes an iron chelating agent in a free form and pharmaceutically acceptable salts thereof. In the Description, the term “iron chelating agent” includes those in the form of hydrochloride salt.

In the Description, the term “transferrin-bound iron” refers to iron ions bound to transferrin. Transferrin is responsible for transport of iron to each tissue through its binding with iron ions. Transferrin receptors are present on the cell surface. When iron-bound transferrin binds to the receptors, it is incorporated into cells by endocytosis, and then iron is released within the cells. Thus, iron is supplied to the cells. Rapidly growing cells have a high demand of iron, so that the importance of transferrin in nutritional supply is high. Accordingly, in recent years, the use of therapeutic methods using strong iron chelating agents capable of chelating iron in vivo has been attempted for cancer therapy. However, these chelating agents chelate both transferrin-bound iron and biologically unstable iron indiscriminately.

In the Description, the term “polymer” refers to “A molecule with high relative molecular weight, the structure of which essentially comprises the multiple repetition of units derived, substantially or conceptually, from molecules with low relative molecular weight” according to the IUPAC definition. The average molecular weight of a polymer means number average molecular weight unless otherwise specified. In the Description, molecules other than polymers are referred to as “low-molecular-weight molecule(s)”.

In the Description, the term “biologically unstable iron” refers to iron ions not bound to transferrin. Hence, examples of biologically unstable iron do not include, iron bound to transferrin (iron ions present in transferrin-iron complex: transferrin-bound iron), storage iron present as ferritin in the liver, spleen and bone marrow, hemoglobin composed of four heme molecules (porphyrin complex containing iron) and one globin molecule (comprised of four polypeptide chains) contained in erythrocytes, and myoglobin present in muscle in the form of chromoprotein storing oxygen molecules until these molecules are required for metabolism and containing a single heme molecule. It is considered that such iron ions; that is, biologically unstable iron, generally exist in vivo not in a free form, but in a form pairing with anions, or forming chelates with amino acids-peptides. Examples of an anion can include compounds formed via binding with hydroxyl ion (OH⁻), citric acid or the like. Examples of such a form can include, Fe³⁺.3(OH⁻) and a hydroxy-citrate-(Cit)complex (FeCitOH⁻). Biologically unstable iron is considered to be iron unnecessary for cell survival, but has been revealed in recent years to harm the body.

In the Description, the expression “having selectivity for biologically unstable iron rather than for transferrin-bound iron” (hereinafter, may also expressed as “biologically unstable iron-selective”) means that, when used for a chelating agent, the chelating agent has selectivity or specificity for biologically unstable iron higher than that for transferrin-bound iron. The expression “having selectivity for biologically unstable iron rather than for transferrin-bound iron” can mean that, for example, 30% or more, 35% or more, 40% or more, 45% or more or 50% or more of biologically unstable iron can be adsorbed and removed, while 80% or more, 85% or more, 90% or more, or 95% or more of transferrin-bound iron is maintained after administration.

In the Description, the expression “iron chelating agent having selectivity for biologically unstable iron rather than for transferrin-bound iron” (hereinafter, may also expressed as “the iron chelating agent of the present invention”) means that the iron chelating agent has a substrate and a chelating moiety, wherein the substrate and the chelating moiety are linked through a bond non-cleavable in vivo (for example, —NH—CH₂— bond). Examples of the iron chelating agent of the present invention include, but are not particularly limited to, an iron chelating agent having a substrate selected from the group consisting of a polymer backbone, glucosamine, and histidine, and these polymers (for example, homopolymers); and an aromatic ring bonded to the substrate through an —NH—CH₂— bond, wherein the aromatic ring has one or two first functional groups, which are each a hydroxyl group; and one or two second functional groups selected from a hydroxyl group, a carboxylic acid group, and a functional group represented by formula (Ia):

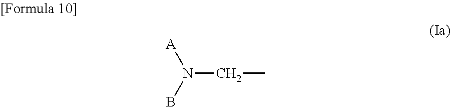

wherein A is —CH₃, —CH₂—CH₃, —CH₂—C(H₅, —CH₂—C₅H₄N, or —CH₂—COOH; and B is —CH₂—COOH. The second functional group is located in the ortho position relative to at least one of the first functional groups, and this structure enables biologically unstable iron-selective chelation of iron ions.

In one preferred embodiment, a biologically unstable iron-selective iron chelating site is an aromatic ring having two hydroxyl groups located in the ortho position, and coordinate bonds are formed, so that a stable five-membered coordination geometry including an iron ion is formed. For example, in one embodiment, the biologically unstable iron-selective iron chelating agent site has a structure represented by the following formula (Ib) in the coordination state.

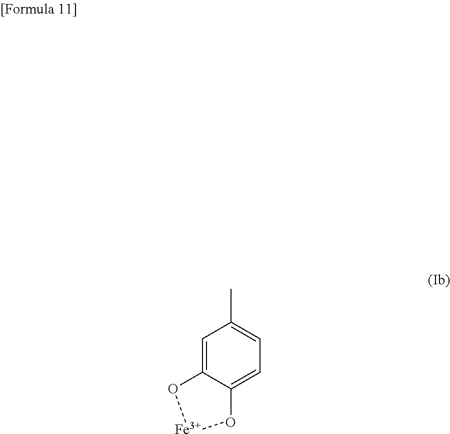

In one preferred embodiment, the biologically unstable iron-selective iron chelating site may be an aromatic ring having three or more hydroxyl groups as long as two hydroxyl groups are located in the ortho position, and coordinate bonds are formed, so that a stable five-membered chelate ring including an iron ion is formed. For example, in one embodiment, the biologically unstable iron-selective iron chelating agent site has a structure represented by the following formula (Ic) in the coordination state.

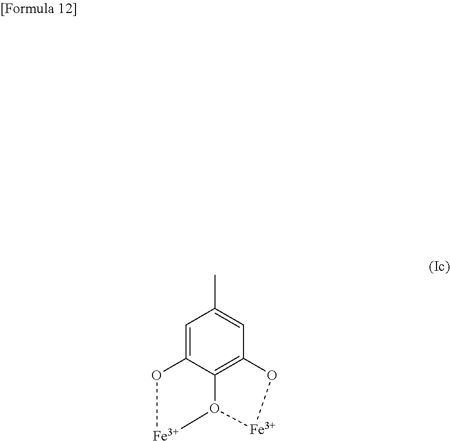

The following formula (IV) represents a structure that is an example of the coordination state of the biologically unstable iron-selective polymeric iron chelating agent having such an iron chelating site. In the chemical formula, the wavy line indicates the polymer backbone. Examples of the types of polymer backbones include linear and branched backbones, those having side chains, or those having three-dimensional network structures. Typically, “n” represents an arbitrary integer, and can range from 100 to 2,000,000, 1,000 to 1,000,000, or 2,000 to 1,000,000. Note that in the formula, the polymer is illustrated for convenience as having a complete repeating structure, but the illustration is intended to include those in which a plurality of iron chelating sites are introduced randomly into the polymer backbone (the same applies to the following descriptions). The iron chelating agent represented by formula (IV) can preferably be hydrochloride salt.

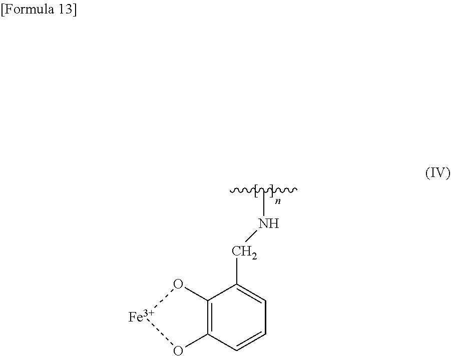

In another preferred embodiment, the biologically unstable iron-selective iron chelating site is an aromatic ring having one hydroxyl group and one carboxylic acid group located in the ortho position, and coordinate bonds are formed so that a stable, six-membered chelate ring including an iron ion is formed. For example, in one embodiment, the biologically unstable iron-selective iron chelating agent site has a structure represented by the following formula (Id) in the coordination state.

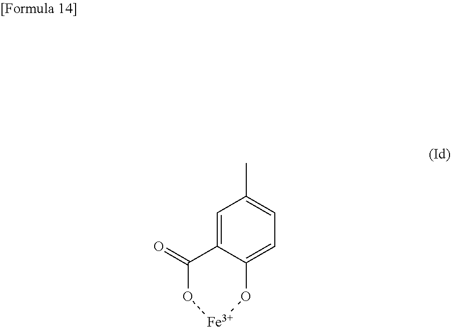

In still another preferred embodiment, the biologically unstable iron-selective iron chelating site is an aromatic ring having one hydroxyl group and a functional group represented by formula (Ia) located in the ortho position, and is capable of chelating biologically unstable iron with a stable coordinate structure composed of one five-membered chelate ring and one six-membered chelate ring. An example of the coordinate structure is represented by the following formula (II).

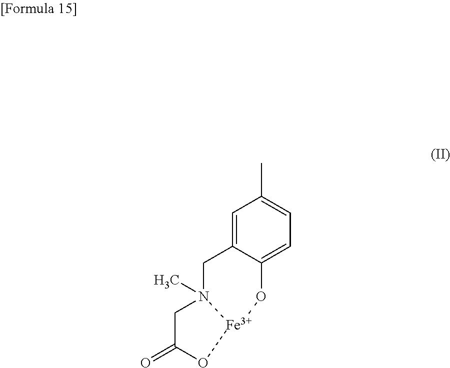

In still another preferred embodiment, the biologically unstable iron-selective iron chelating site is an aromatic ring having one hydroxyl group and two functional groups represented by formula (I) located in positions ortho to both sides of the hydroxyl group. This makes it possible to increase the amount of biologically unstable iron that can be chelated per iron chelating site. An example of the coordinate state of such biologically unstable iron-selective iron chelating site is represented by the following formula (III).

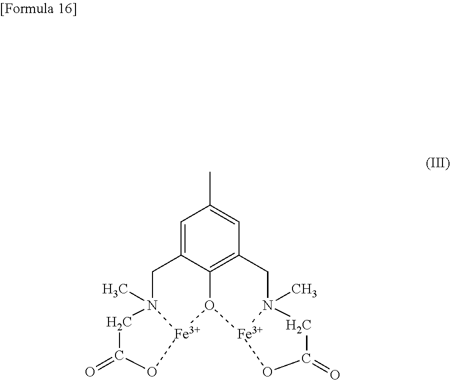

The polymer backbone represents a polymer molecule which is capable of functioning as a carrier by forming a covalent bond with an aromatic ring that acts as a biologically unstable iron-selective iron chelating site. Examples of polymer backbones preferably used in the present invention include (1) known water-insoluble polymers such as polyethylene, polypropylene, polystyrene, polyvinylidene fluoride, polytetrafluoroethylene or polyethylene terephthalate, (2) water-soluble polymers having amino groups such as poly(allylamine) or polyethyleneimine which can be insolubilized by crosslinking, and (3) water-insoluble natural polymers having primary amino groups.

In the polymeric chelating agent of the present invention, the polymer backbone is bonded to an aromatic ring directly through an —NH—CH₂— bond, wherein the aromatic ring has one or two first functional groups, which are each a hydroxyl group; and one or two second functional groups selected from a hydroxyl group, a carboxylic acid group, and a functional group represented by formula (I), and wherein the second functional group is located in the ortho position relative to at least one of the first functional groups. Bonding of the polymer backbone to the aromatic ring directly through the —NH—CH₂— bond as described above is preferred, since not only the —NH—CH₂— bond itself has excellent hydrolysis resistance compared with that of an —NH—CO— bond or a —CO—O— bond, but also the —NH—CH₂— bond allows a polymeric chelating agent to be uniformly generated in a hydrophilic solvent compatible with the resulting polymeric chelate, unlike —CH₂— or —CH₂—CH₂— bond, etc. In one embodiment, the polymer backbone of the polymeric chelating agent of the present invention is chitosan. In one embodiment, the polymeric chelating agent of the present invention is in the form of hydrochloride salt. In one embodiment, the polymeric chelating agent of the present invention has chitosan as the polymer backbone and is in the form of hydrochloride salt.

In order to produce the polymeric iron chelating agent of the present invention, amino groups are first introduced into the polymer backbone or a polymer backbone having amino groups are prepared. A natural polymer having a primary amino group such as chitosan can be used as a substrate of the polymeric iron chelating agent of the present invention. Chitosan can be particularly preferably used since it contains numerous primary amino groups available for the introduction of iron chelating sites through an —NH—CH₂— bond (i.e. a large number of primary amino groups per unit weight of polymer).

The polymeric iron chelating agent of the present invention is, in an embodiment, soluble in water, and a water-soluble polymer backbone can be used as a starting material.

Next, the above polymer backbone, either having amino groups or amino groups through introduction thereof, is reacted with an aldehyde derivative of the aromatic ring serving as an iron chelating site, then the resultant is reduced to form an —NH—CH₂— bond between the polymer backbone and the aromatic ring. The aldehyde derivative of the aromatic ring is a compound having an aldehyde group at the position on the aromatic ring where the polymer is to be bound. In an embodiment, the polymeric chelating agent of the present invention is in the form of hydrochloride salt. In an embodiment, the polymeric chelating agent of the present invention is in the form of hydrochloride salt, wherein the substrate is glucosamine or histidine or an oligomer thereof.

The polymeric iron chelating agent of the present invention can be produced as follows, for example.

1) An amino group of chitosan is reacted with an aldehyde group of 2,3-dihydroxybenzaldehyde by reacting chitosan with 2,3-dihydroxybenzaldehyde in a mixed solvent consisting of 5% acetic acid and methanol. Sodium borohydride is slowly added to the resulting gelatinous solution until a crystalline precipitate is formed. The thus obtained compound is reduced. From this reaction, a polymeric iron chelating agent can be obtained, wherein chitosan and an aromatic ring having two hydroxyl groups located in the ortho position and being capable of chelating iron are bonded through an —NH—CH₂— bond (view from the chitosan side).

2) A polymeric iron chelating agent capable of capturing two iron ions with two five-membered rings and two six-membered rings can be obtained by employing N,N′-(2-hydroxy-5-formyl-1,3-dixylene)bis(N-(methyl)-glycine) in place of 2,3-dihydroxy-benzaldehyde in 1) above (which can be synthesized by reacting para-hydroxybenzaldehyde and N-methylglycine in an aqueous formaldehyde solution according to the method described in Bruce P. Murch, et al., J. Am. Chem. Soc., 1985, 107 (23), pp. 6728-6729) (see formula (III)).

The iron chelating agent of the present invention is not required to be a polymer, and can be a chelating agent prepared by introducing an iron chelating site into a substrate other than a polymer, such as a low-molecular-weight substrate (for example, a low-molecular-weight biomolecule). Examples of a low-molecular-weight substrate include biomolecules having amino groups such as glucosamine and histidine and oligomers formed via polymerization of these 2 to several (for example, 2) molecules.

The iron chelating agent of the present invention can be obtained by introducing an amino group into a substrate, or reacting an aldehyde derivative of an aromatic ring serving as an iron chelating site with an amino group of a substrate, and then reducing the resultant, to form an —NH—CH₂— bond between the polymer backbone and the aromatic ring, for example. The aldehyde derivative of the aromatic ring is a compound having an aldehyde group at a position on the aromatic ring, where the polymer is to be bound.

The iron chelating agent of the present invention is selective for biologically unstable iron, and can be particularly suitably used for removal of biologically unstable iron.

The iron chelating agent of the present invention is coordinated with iron ions to form a complex (iron complex or iron chelate), which has a characteristic absorption wavelength (absorbance wavelength) that differs from the absorbance wavelength of iron ions or iron chelating agents.

Specifically, the effects of chelating iron ions of the iron chelating agent of the present invention can be confirmed by adding the iron chelating agent of the present invention to a solution containing iron ions, and then after completion of the reaction (chelating reaction), comparing the color developed by the iron chelating agent; that is, the color before the chelating reaction with the color after the chelating reaction, for example.

As a solvent for a solution or a suspension solution containing iron ions, which is to be used upon chelating of iron ions, Dulbecco's Phosphate-Buffered Saline (D-PBS(−)) or pure water (namely, “Milli-Q water” produced by, for example, “Milli-Q” ultrapure water purification system manufactured by Millipore Corporation) can be used. One type of solvent may be used or two or more types thereof can be used in combination.

Regarding a method for capturing iron ions, iron ions can be captured using the iron chelating agent of the present invention. The polymeric iron chelating agent of the present invention has extremely high capacity of chelating biologically unstable iron (particularly, trivalent iron ions), and is capable of selectively capturing biologically unstable iron, to be able to effectively reduce the amount of biologically unstable iron in the system.

According to the present invention, the iron chelating agent of the present invention exhibits antitumor effects. Biologically unstable iron is considered to be unnecessary for living bodies. However, the results of Examples revealed that the removal of biologically unstable iron leads to antitumor effects. Biologically unstable iron has a bad effect on living bodies. Hence, the selective removal of biologically unstable iron not only can lower such a bad effect due to biologically unstable iron, but also has antitumor effects on cancer patients, and, therapeutic effects on infectious diseases of patients with the infectious diseases.

Therefore, according to the present invention, provided is the pharmaceutical composition for use in treatment of cancer comprising an iron chelating agent, which has selectivity for biologically unstable iron rather than for transferrin-bound iron.

As the iron chelating agent having selectivity for biologically unstable iron rather than for transferrin-bound iron, for example, the above-described iron chelating agent of the present invention can be used.

In an embodiment, the iron chelating agent of the present invention contained in the above pharmaceutical composition can be an iron chelating agent having an aromatic ring having a structure represented by the following formula (Ie).

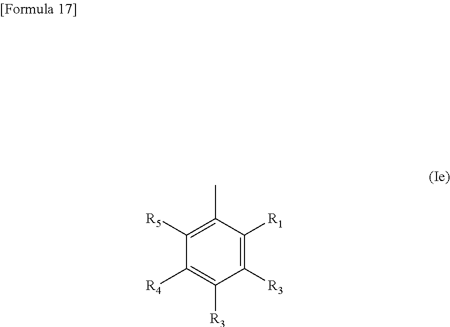

wherein any one of R₁ to R₅ is OH; the ring has at least OH or COOH in the ortho position relative to the OH; and the other groups are selected from H, OH, COOH, CH₃, and —N(CH₃)—CH₂—COOH.

In an embodiment, the iron chelating agent of the present invention contained in the above pharmaceutical composition contains a chitosan backbone as a substrate. In an embodiment, the iron chelating agent of the present invention contained in the above pharmaceutical composition contains glucosamine as a substrate. Glucosamine can be in an equilibrium state with a closed ring structure and an open ring structure in an aqueous solution. In an embodiment, the iron chelating agent of the present invention contained in the above pharmaceutical composition contains histidine as a substrate. In an embodiment, the above glucosamine or histidine may be incorporated as a monomer unit into a polymer.

In an embodiment, the iron chelating agent of the present invention contained in the above pharmaceutical composition can be an iron chelating agent having the following structure:

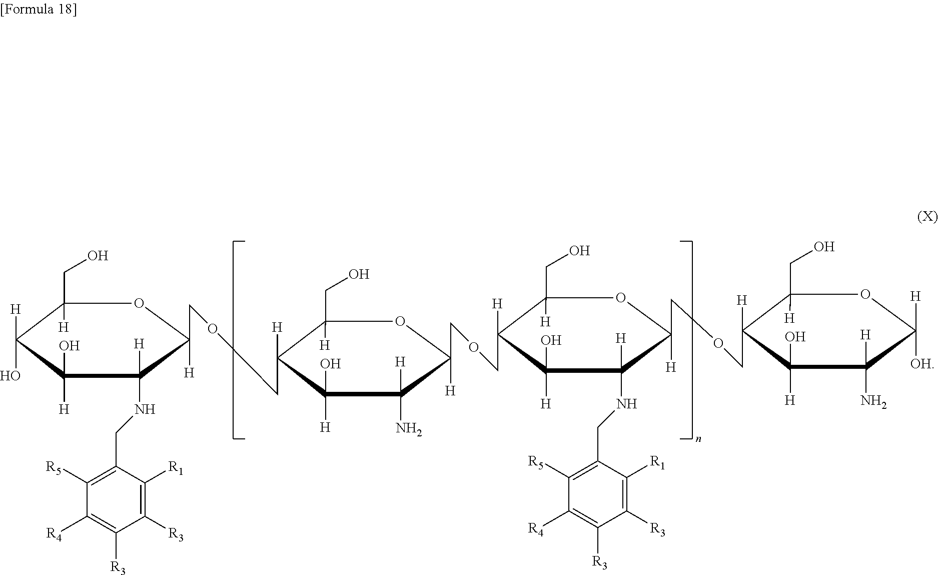

The above formula (X) depicts how the chelating sites represented by formula (Ie) are directly bonded to the chitosan backbone through an —NH—CH₂— bond. In the above formula (X), monomer units to which the chelating sites are linked and monomer units to which no chelating sites are linked are regularly described for convenience in description, but these sites may be randomly located. In the above formula (X), the amount of the chelating sites linked to monomer units of chitosan can be adjusted stoichiometrically. For example, 30% or more, 40% or more, 50% or more, 60% or more, 70% or more, 80% or more, 90% or more, or 95% or more, or substantially 100% of the monomer units may be modified by the chelating sites. Note that the compound of formula (X) can be in the form of hydrochloride salt in a preferred embodiment. In the above formula (X), the average molecular weight of chitosan can range from 20,000 to 80,000, 30,000 to 70,000, or 40,000 to 60,000, for example.

In an embodiment, the iron chelating agent of the present invention contained in the above pharmaceutical composition has any one of chelating sites selected from cate-2, cate-3 and carb-2 represented by the following chemical formula (If). In an embodiment, the iron chelating agent of the present invention contained in the above pharmaceutical composition contains a chitosan backbone as a substrate, and has any one of chelating sites selected from cate-2, cate-3 and carb-2 represented by the following chemical formula. In an embodiment, the iron chelating agent of the present invention contained in the above pharmaceutical composition contains histidine as a substrate, and has any one of chelating sites selected from cate-2, cate-3 and carb-2 represented by the following chemical formula. In an embodiment, the iron chelating agent of the present invention contained in the above pharmaceutical composition contains glucosamine as a substrate, and has any one of chelating sites selected from cate-2, cate-3 and carb-2 represented by the following chemical formula. In these embodiments, substrates and chelating sites may be linked to each other via —NH—CH₂—.

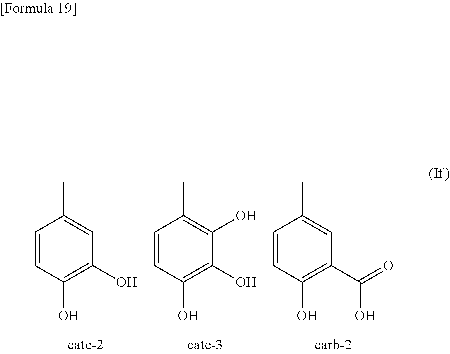

Here, cate-2 in the above formula (If) is in a case when R₃ is OH, R₂ or R₄ is OH in the above formula (Ie), cate-3 in (If) is in a case when R₁ to R₃ are each OH or R₃ to R₅ are each OH in the above formula (Ie), and carb-2 in (If) is in a case when R₃ is OH, and R₂ or R₄ is COOH.

In an embodiment, in the iron chelating agent of the present invention, the chelating site can be cate-2 in formula (X). In an embodiment, in the iron chelating agent of the present invention, the chelating site can be cate-3 in formula (X). In the iron chelating agent of the present invention, the chelating site can be carb-2 in formula (X).

In an embodiment, the iron chelating agent of the present invention contained in the above pharmaceutical composition can be represented by the following formula:

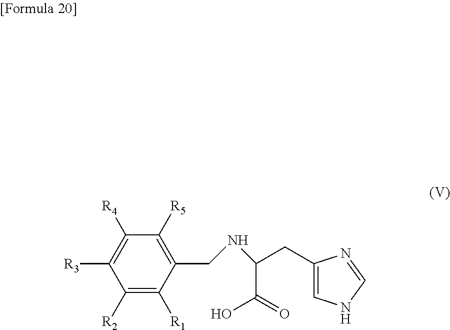

wherein any one of R₁ to R₅ is OH; the ring has at least OH or COOH in the ortho position relative to the OH; and the other groups are selected from H, OH, COOH, CH₃—, and —N(CH₃)—CH₂—COOH. In another embodiment, the iron chelating agent of the present invention contained in the above pharmaceutical composition wherein any one of R₁ to R₅ is OH, and has a group selected from at least OH and COOH in the ortho position relative to the above OH. The above iron chelating agent is preferably an acid addition salt (preferably, hydrochloride salt). In an embodiment, the following formula:

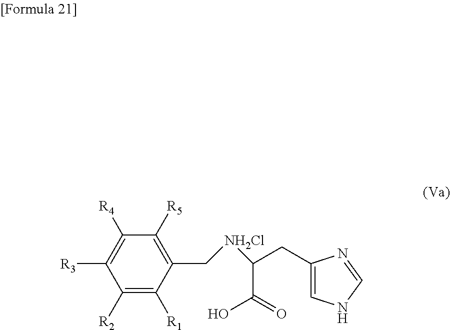

wherein any one of R₁ to R₅ is OH; the ring has at least OH or COOH in the ortho position relative to the OH; and the other groups are selected from H, OH, COOH, CH₃, and —N(CH₃)—CH₂—COOH, can be used as the iron chelating agent.

In an embodiment, the iron chelating agent of the present invention contained in the above pharmaceutical composition comprises an aromatic ring bonded to an N atom of glucosamine through —CH₂—, wherein the aromatic ring is represented by the following formula:

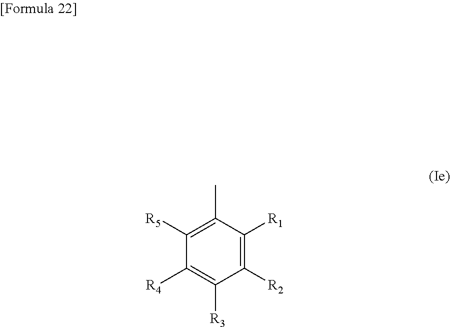

wherein any one of R₁ to R₅ is OH; the ring has at least OH or COOH in the ortho position relative to the OH; and the other groups are selected from H, OH, COOH, CH₃, and —N(CH₃)—CH₂—COOH. Further, in an embodiment, the iron chelating agent of the present invention contained in the above pharmaceutical composition can be represented by the following formula:

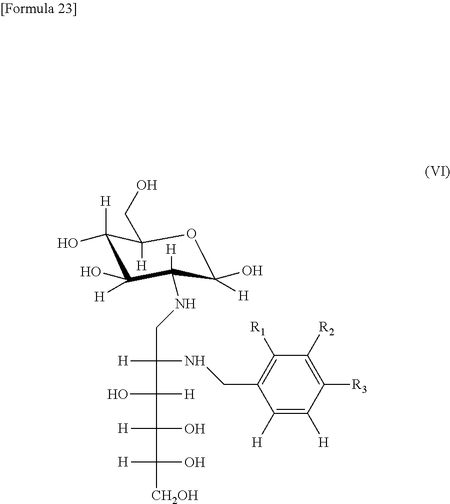

wherein R₁ to R₃ are as defined above. Note that formula (VI) is an embodiment in which R₄ and R₅ are each H in aromatic ring (Ie), and the substrate is glucosamine. In an embodiment, R₁ is H, R₂ is OH, and R₃ is OH. In an embodiment, R₁ to R₃ are each OH. The above iron chelating agent is preferably an acid addition salt (preferably hydrochloride salt). In an embodiment, the following formula:

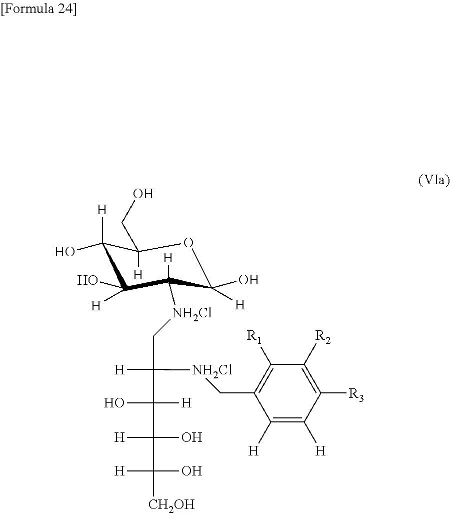

wherein R₁ to R₃ are as defined above, can be used as the iron chelating agent. In an embodiment, R₁ is H; R₂ is OH; and R₃ is OH. In an embodiment, R₁ to R₃ are each OH.

The above pharmaceutical composition is effective for all cancer types and examples thereof, for which the pharmaceutical composition can be effective, include, but are not particularly limited to, lung cancer, large bowel cancer, breast cancer, ovarian cancer, liver cancer, pancreas cancer, uterine cancer, oral cancer, epithelial cancer (for example, squamous cell carcinoma), and leukemia.

According to the present invention, the iron chelating agent having selectivity for biologically unstable iron rather than for transferrin-bound iron has antimicrobial effects. Therefore, according to the present invention, provided is an antimicrobial agent (or pharmaceutical composition for use in treatment of bacterial infection) comprising the iron chelating agent having selectivity for biologically unstable iron rather than for transferrin-bound iron.

In the antimicrobial agent of the present invention, as the iron chelating agent having selectivity for biologically unstable iron rather than for transferrin-bound iron, the iron chelating agent of the present invention can be used. An iron chelating agent same as that described for the above pharmaceutical composition for use in treatment of cancer can be used as a preferable iron chelating agent.

The antimicrobial agent of the present invention can be effective against Staphylococcus aureus (S. aureus), Pseudomonas aeruginosa (P. aeruginosa), Candida albicans (C. albicans), and Streptococcus mutans (S. mutans), as well as, periodontopathic bacteria, such as Porphyromonas gingivalis (P. gingivalis) and Aggregatibacter actinomycetemcomitans (A. actinomycetemcomitans), for example, but the examples thereof are not particularly limited thereto. Of these, S. mutans, P. gingivalis, and A. actinomycetemcomitans may be generically referred to as oral bacteria. In addition, P. gingivalis, and A. actinomycetemcomitans may be generically referred to as periodontopathic bacteria.

In particular, the iron chelating agent of the present invention having chitosan as a substrate exhibits strong antimicrobial effects on these bacteria. Particularly the iron chelating agent of the present invention having chitosan as a substrate exhibits strong antimicrobial effects on S. aureus and C. albicans.

According to the present invention, provided is a pharmaceutical composition (or antimicrobial agent for use against S. aureus and/or C. albicans) for use in treatment of S. aureus and/or C. albicans in a subject infected with S. aureus and/or C. albicans, which comprises an iron chelating agent, wherein

a substrate is chitosan, and an aromatic ring has any one of chelating sites selected from cate-2, cate-3 and carb-2 (preferably has cate-2). Here, the substrate and the chelating site may be linked to each other via —NH—CH₂—.

According to the present invention, provided is a pharmaceutical composition (or an antimicrobial agent for use against S. aureus) for use in treatment of S. aureus in a subject infected with S. aureus, comprising an iron chelating agent, wherein

a substrate is chitosan, and an aromatic ring has any one of chelating sites selected from cate-2, cate-3 and carb-2 (preferably has cate-2 or carb-2). In a preferred embodiment, the substrate and the chelating site may be linked to each other via —NH—CH₂—.

According to the present invention, provided is a pharmaceutical composition (or an antimicrobial agent for use against S. aureus) for use in treatment of S. aureus in a subject infected with S. aureus, comprising an iron chelating agent, wherein

a substrate is glucosamine and an aromatic ring has any one of chelating sites selected from cate-2, cate-3 and carb-2 (preferably has cate-2 or cate-3). In a preferred embodiment, the substrate and the chelating site may be linked to each other via —NH—CH₂—.

According to the present invention, provided is a pharmaceutical composition (or an antimicrobial agent for use against P. aeruginosa) for use in treatment of P. aeruginosa infection in a subject infected with P. aeruginosa, comprising an iron chelating agent, wherein

a substrate is glucosamine and an aromatic ring has any one of chelating sites selected from cate-2, cate-3 and carb-2 (preferably has cate-3). In a preferred embodiment, the substrate and the chelating site may be linked to each other via —NH—CH₂—.

According to the present invention, provided is a pharmaceutical composition (or an antimicrobial agent for use against C. albicans) for use in treatment of C. albicans in a subject infected with C. albicans, comprising an iron chelating agent, wherein

a substrate is chitosan, and an aromatic ring has any one of chelating sites selected from cate-2, cate-3 and carb-2 (preferably has cate-2). In a preferred embodiment, the substrate and the chelating site may be linked to each other via —NH—CH₂—.

According to the present invention, provided is a pharmaceutical composition (or an antimicrobial agent for use against C. albicans) for use in treatment of C. albicans in a subject infected with C. albicans, comprising an iron chelating agent, wherein

a substrate is glucosamine and an aromatic ring has cate-2. In a preferred embodiment, the substrate and the chelating site may be linked to each other via —NH—CH₂—.

According to the present invention, provided is a pharmaceutical composition (or an antimicrobial agent for use against S. aureus and/or C. albicans) for use in treatment of S. aureus and/or C. albicans in a subject infected with S. aureus and/or C. albicans, comprising an iron chelating agent, wherein

a substrate is histidine and an aromatic ring has any one of chelating sites selected from cate-2, cate-3 and carb-2 (preferably has cate-2). In a preferred embodiment, the substrate and the chelating site may be linked to each other via —NH—CH₂—.

According to the present invention, provided is a pharmaceutical composition (or an antimicrobial agent for use against periodontopathic bacteria) for use in treatment of periodontopathic bacteria in a subject infected with periodontopathic bacteria (for example, S. mutans or P. gingivalis), comprising an iron chelating agent, wherein

a substrate is chitosan and an aromatic ring has any one of chelating sites selected from cate-2, cate-3 and carb-2 (for example, has cate-2). In a preferred embodiment, the substrate and the chelating site may be linked to each other via —NH—CH₂—.

According to the present invention, provided is a pharmaceutical composition (or an antimicrobial agent for use against oral bacteria) for use in treatment of oral bacteria in a subject infected with oral bacteria (for example, S. mutans, P. gingivalis, or A. actinomycetemcomitans) such as periodontopathic bacteria (for example, P. gingivalis or A. actinomycetemcomitans), comprising an iron chelating agent, wherein

a substrate is glucosamine and an aromatic ring has any one of chelating sites selected from cate-2, cate-3 and carb-2 (for example, has cate-2 or cate-3). In a preferred embodiment, the substrate and the chelating site may be linked to each other via —NH—CH₂—.

According to the present invention, provided is a pharmaceutical composition (or an antimicrobial agent for use against periodontopathic bacteria) for use in treatment of periodontopathic bacteria in a subject infected with oral bacteria (for example, S. mutans, P. gingivalis, or A. actinomycetemcomitans) such as periodontopathic bacteria (for example, S. mutans or P. gingivalis), comprising an iron chelating agent, wherein

a substrate is histidine and an aromatic ring has any one of chelating sites selected from cate-2, cate-3 and carb-2 (for example, has cate-3). In a preferred embodiment, the substrate and the chelating site may be linked to each other via —NH—CH₂—.

According to the present invention, the iron chelating agent having selectivity for biologically unstable iron rather than for transferrin-bound iron can suppress viral infection and can suppress viral cytocidal effects. Therefore, according to the present invention, the iron chelating agent can be used for treating viral infection, such as prevention or treatment of viral infection. Therefore, according to the present invention, provided is a pharmaceutical composition for use in prevention or treatment of viral infection, comprising the iron chelating agent having selectivity for biologically unstable iron rather than for transferrin-bound iron. According to the present invention, preferably the chelating agent of the present invention can be used as the iron chelating agent having selectivity for biologically unstable iron rather than for transferrin-bound iron. In an embodiment, the chelating agent of the present invention can be an iron chelating agent having a substrate and a chelating site having an aromatic ring structure represented by formula (Ie), for example. In an embodiment, the substrate can be one or more substrates selected from the group consisting of chitosan, glucosamine, and histidine. In an embodiment, the chelating site is any one of chelating sites selected from cate-2, cate-3 and carb-2 represented by formula (If). In an embodiment, the chelating site has cate-3. In an embodiment, the substrate and the chelating site may be linked to each other through —NH—CH₂— or directly via —NH—CH₂—.

In an embodiment of the present invention, the iron chelating agent having selectivity for biologically unstable iron rather than for transferrin-bound iron has a substrate selected from the group consisting of glucosamine and histidine; and a chelating site that can be cate-3. In this embodiment, the substrate and the chelating site may also be linked via a non-cleavable bond. In an embodiment, the base and the chelating site may be linked to each other through —NH—CH₂— or directly via —NH—CH₂—.

A virus, against which the iron chelating agent having selectivity for biologically unstable iron rather than for transferrin-bound iron is effective, can be an influenza virus, for example.

The pharmaceutical composition or the antimicrobial agent of the present invention may also contain an excipient. The pharmaceutical composition or the antimicrobial agent of the present invention can be a formulation suitable for parenteral administration such as intravenous administration, intramuscular administration, and subcutaneous administration. The pharmaceutical composition for treatment of cancer can also be a formulation suitable for intratumoral administration. An antimicrobial agent for use against bacteria causing dental caries and periodontopathic bacteria can also be a formulation suitable for intraoral administration.

According to another aspect of the present invention, provided is the iron chelating agent of the present invention for use in a method for treating cancer in a subject in need thereof. According to another aspect of the present invention, provided is the iron chelating agent of the present invention for use in a method for treating infectious disease in a subject in need thereof.

According to another aspect of the present invention, provided is the use of the iron chelating agent of the present invention in manufacture of a pharmaceutical composition for use in treatment of cancer. According to another aspect of the present invention, provided is the use of the iron chelating agent of the present invention in manufacture of a pharmaceutical composition for use in treatment of bacterial infection.

As used herein, any embodiment represented with the expression “comprising” encompass embodiments represented with the expression “essentially consisting of” as well as embodiments represented with the expression “consisting of”.

The contents of all patents and references cited explicitly herein are incorporated herein by reference in their entirety.

The present invention is described more specifically with reference to examples as follows, but the present invention is not limited to the following examples.

### EXAMPLES

### Example 1: Preparation of Chelating Agent Selective for Biologically Unstable Iron Rather than for Transferrin-Bound Iron

In the examples, on the basis of the findings disclosed in WO2012/096183, chelating agents selective for biologically unstable iron rather than for transferrin-bound iron were prepared. Note that all the prepared chelating agents were in the form of hydrochloride salt.

Specifically, the chelating agents selective for biologically unstable iron rather than for transferrin-bound iron were prepared by introducing the chelating agent sites in Table 2 into the substrates in Table 1, to link aldehyde groups and the amino groups of substrates, respectively, by the method described in WO2012/096183 and WO2016/052488.

TABLE 1
Substrate for introduction of chelating agent

| | | Molecular | | Product |
|---|---|---|---|---|
| Substrate | Reagent used | weight | Manufacturer | number |
| Chitosan | Daichitosan | 40,000- | Dainichiseika | KRM-12007 |
| | (chitosan powder) | 54,000 | Color & Chemicals | |
| | | | Mfg Co., Ltd. | |
| Glucosamine | D-Glucosamine | 215.63 | Nacalai Tesque | 16802-94 |
| | hydrochloride salt | | Inc. | |
| Histidine | L-histidine | 155.15 | Nacalai Tesque | 18116-34 |
| | | | Inc. | |

TABLE 2
Portion of chelating agent to be introduced

| Chelating | | Molecular | | Product |
|---|---|---|---|---|
| site | Reagent used | weight | Manufacturer | number |
| cate-2 | 3,4- | 138.12 | Tokyo Chemical | D0566 |
| | Dihydroxy- | | Industry Co., Ltd. | |
| | benzaldehyde | | | |
| cate-3 | 2,3,4- | 154.12 | Tokyo Chemical | T2158 |
| | Trihydroxybenz- | | Industry Co., Ltd. | |
| | aldehyde | | | |
| carb-2 | 5-Formyl | 166.13 | Tokyo Chemical | F0400 |
| | salicylic acid | | Industry Co., Ltd. | |

The structure of each chelating site extending from the substrate is as follows.

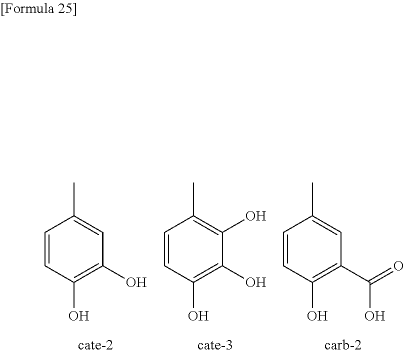

TABLE 3
Examples of chelating agent prepared

| Name | Substrate | Chelating site |
|---|---|---|
| Test group 1 | Chitosan | cate-2 |
| Test group 2 | Chitosan | cate-2* |
| Test group 3 | Chitosan | cate-3 |
| Test group 4 | Chitosan | carb-2 |
| Test group 5 | Glucosamine | cate-2 |
| Test group 6 | Glucosamine | cate-3 |
| Test group 7 | Glucosamine | carb-2 |
| Test group 8 | Histidine | cate-2 |
| Test group 9 | Histidine | carb-2 |
| Test group 10 | Histidine | cate-3 |

*The amount of dihydroxybenzaldehyde introduced was doubled.

Specifically, 600 mg of 3,4-dihydroxybenzaldehyde was dissolved in 100 mL of 5% acetic acid solution (water/methanol=1/1), and then 1.0 g of Daichitosan (Dainichiseika Color & Chemicals Mfg. Co., Ltd.) was added to the solution. In a condition where chitosan was completely dissolved, 2.2 g of sodium hydrogencarbonate was added gradually. After the sodium hydrogencarbonate was completely reacted, NaBH₄ was added gradually (up to about 2 g). As a result, a large amount of white precipitates containing a small amount of yellow crystal was generated. Stirring was continued for a while, and then filtration was performed to give crystal. After washing with methanol, the crystal was suspended in 100 mL of methanol, and then NaBH₄ was added gradually (up to about 0.5 g). When the color of the crystal turned completely white, filtration was performed. The crystal was washed sufficiently with methanol and then dried in a desiccator.

The thus obtained compound (dry sample) was suspended in 100 mL of ethanol, and then 10 mL of concentrated hydrochloric acid was added to the suspension, followed by 1 hour of stirring. The crystal was subjected to suction filtration, washed sufficiently with ethanol, and then dried in vacuum, thereby obtaining the target chelating agent of test group 1. The chelating agents of test groups 2 to 4 were prepared similarly.

Glucosamine hydrochloride salt (Wako Pure Chemical Industries, Ltd., 2.15 g, 0.01 mol) was dissolved in an aqueous solution (10 mL) containing an equivalent amount of NaOH. Forty (40) mL of methanol solution containing 1.38 g (0.01 mol) of 3,4-dihydroxybenzaldehyde was added, and then a small amount of NaBH4 (up to 300 mg) was further added to the solution, followed by reduction. After the resultant was left to stand for 1 hour, the pH of the solution was adjusted to a maximum of pH 4 using dilute hydrochloric acid for concentration. Methanol was added to the resultant, it was left to stand for several hours and then filtered. The filtrate was concentrated and then ethanol was added to the concentrate. Crystal obtained in the form of white precipitate was dried in vacuum, thereby preparing the chelating agent of test group 5. The chelating agents of test groups 6 and 7 were also similarly prepared.

400 mg of NaOH was dissolved in 10 mL of water. 1.55 g of L-histidine (Tokyo Chemical Industry Co., Ltd.) was added, and then 50 mL of methanol was added. Separately, 30 mL of a methanol solution containing 1.54 g (0.01 mol) of 2,3,4-trihydroxybenzaldehyde was prepared, and then mixed with the above histidine solution. Solid NaBH₄ was added gradually to the thus obtained mixture. When the solution became colorless, 1 mol of a hydrochloric acid solution was added gradually to adjust the pH to a maximum of 4. After stirring, the thus generated white precipitate was separated by filtration, and then the solution was concentrated (up to 20 mL). The solution was filtered, ethanol (up to 50 mL) was added to the filtrate, the thus generated white precipitate was filtered and then dried in vacuum, thereby obtaining the chelating agent of test group 10. The chelating agents of test groups 8 and 9 were also similarly prepared.

### Example 2: Examination of Antitumor Effects

Each of the chelating agents prepared in Example 1 was confirmed for the antitumor effects.

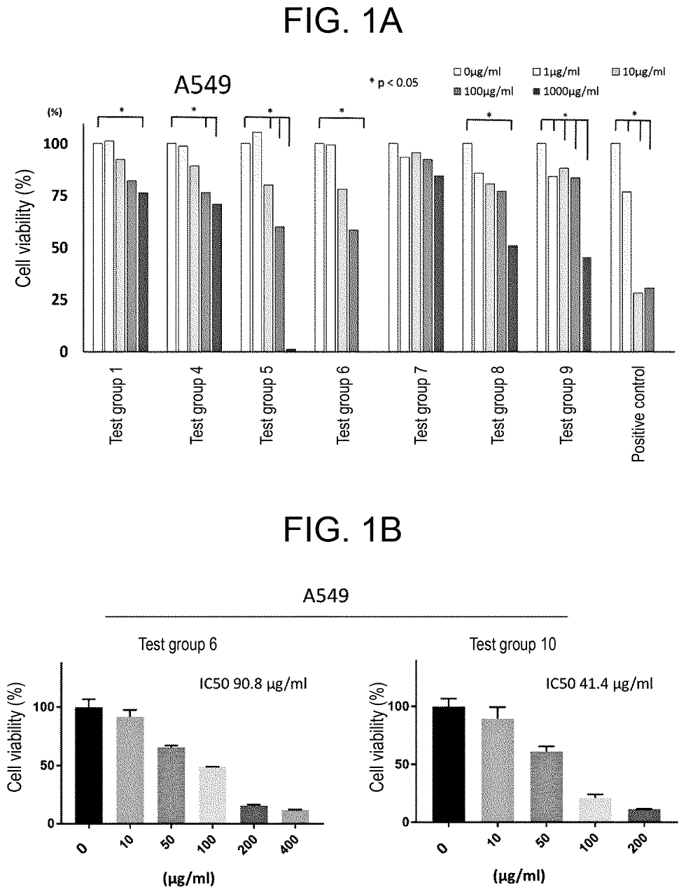

The antitumor effects of the chelating agents of test groups 1 to 10 on each of human lung cancer cell line A549, human liver cancer cell line PLC, and human colon adenocarcinoma cell line HCT116 were confirmed. A549 cells or PLC cells were seeded at 3000 cells/well, and HCT116 cells were seeded at 6000 cells/well. After 24 hours, the fetal bovine serum (FBS) concentration was changed from 10% to 1%, each compound of the test groups 1 to 10 was introduced into medium. Cell viability was confirmed after 48 hours by the Trypan blue method, or, cell viability was confirmed after 48 hours by performing medium exchange and then after 24 hours by performing the XTT method. Deferoxamine mesylate (Desferal (Trademark)) was used as a positive control. Results are as depicted in FIGS. 1A, 1B, 2 and 3.

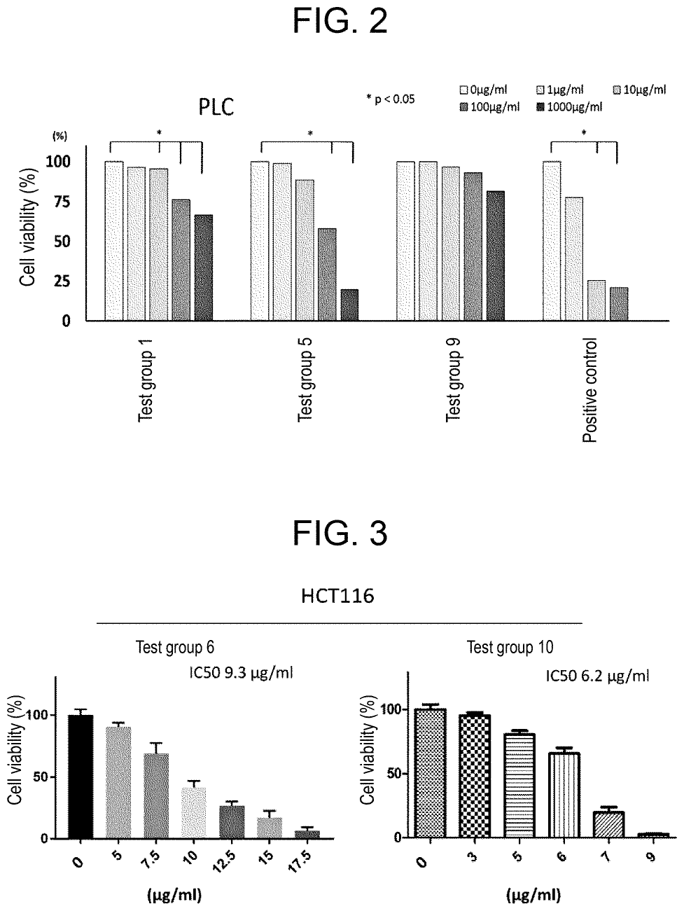

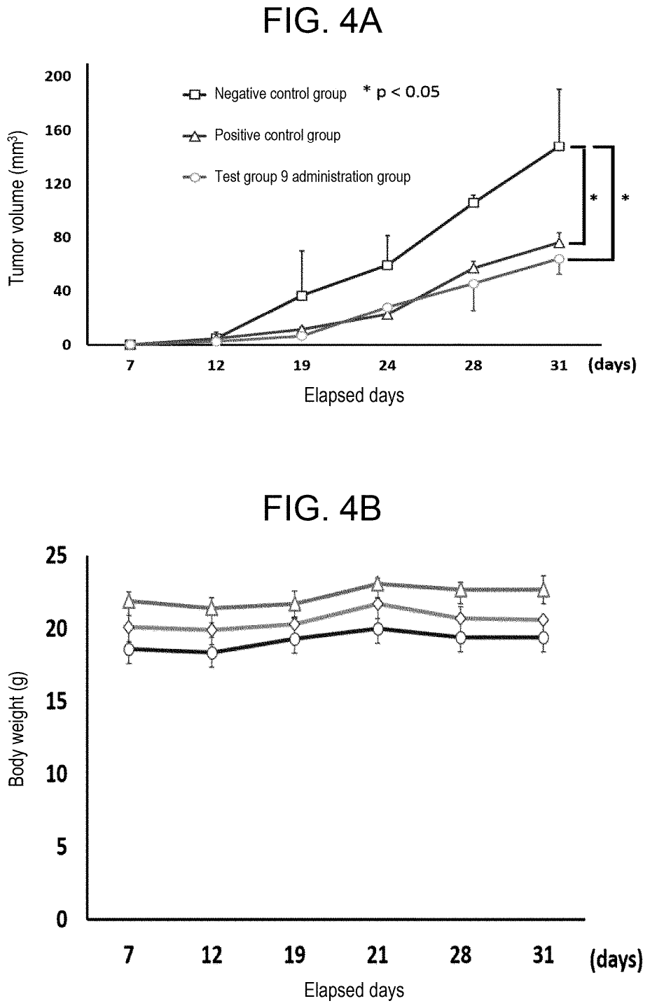

As depicted in FIGS. 1A and 1B, all the tested chelating agents of the test groups were found to exhibit antitumor effects on A549 cell line in a concentration-dependent manner. Further, as depicted in FIG. 2, all the test groups were found to exhibit antitumor effects on PLC cell line in a concentration-dependent manner. Further, as depicted in FIG. 3, all the test groups were found to exhibit antitumor effects on HCT116 cell line in a concentration-dependent manner.

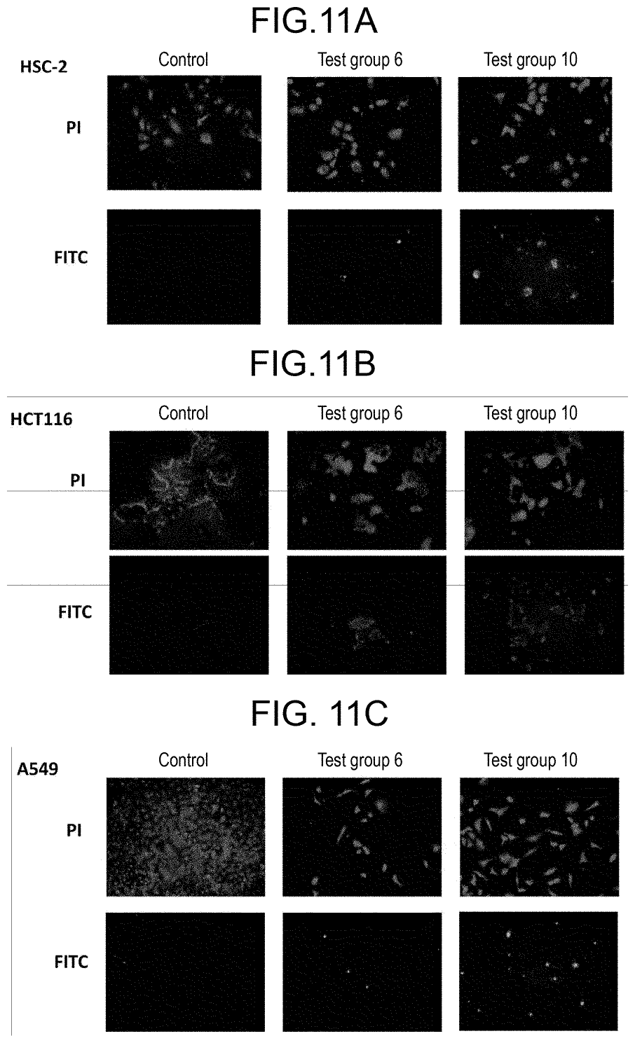

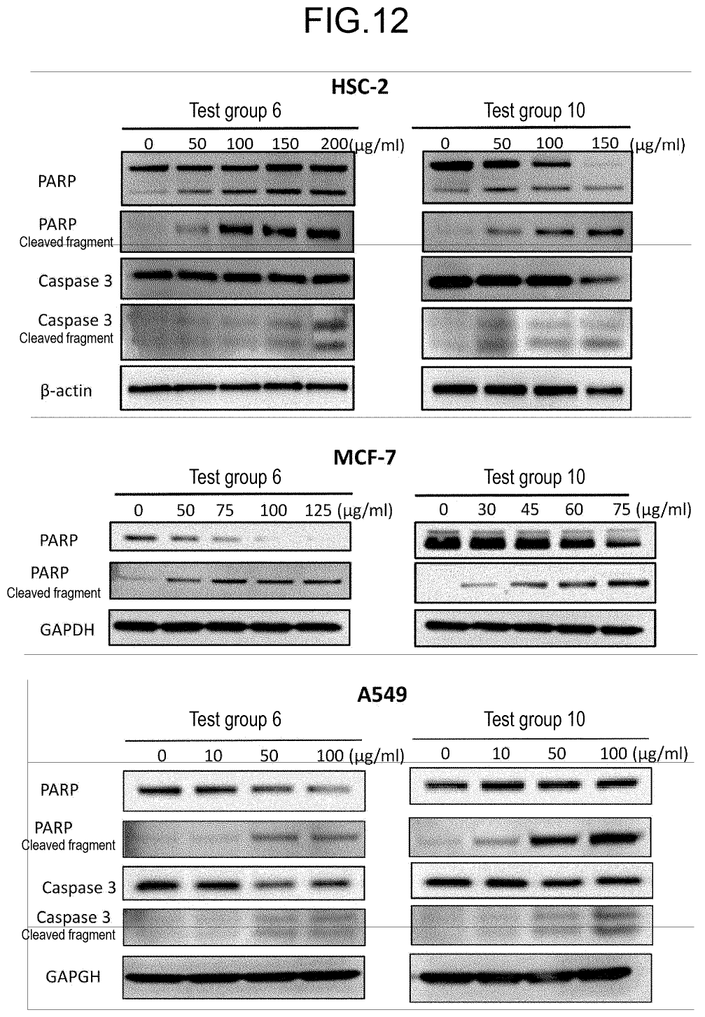

Moreover, similarly to the above experiment on antitumor effects, the antitumor effects of the chelating agent of test group 6 or 10 on each of human breast cancer cell line MCF-7 and human oral squamous cell carcinoma HSC-2 were confirmed. As a result, the chelating agents of test group 6 and test group 10 were each found to also exhibit antitumor effects on both MCF-7 (see FIG. 9) and HSC-2 (see FIG. 10) in a concentration-dependent manner.

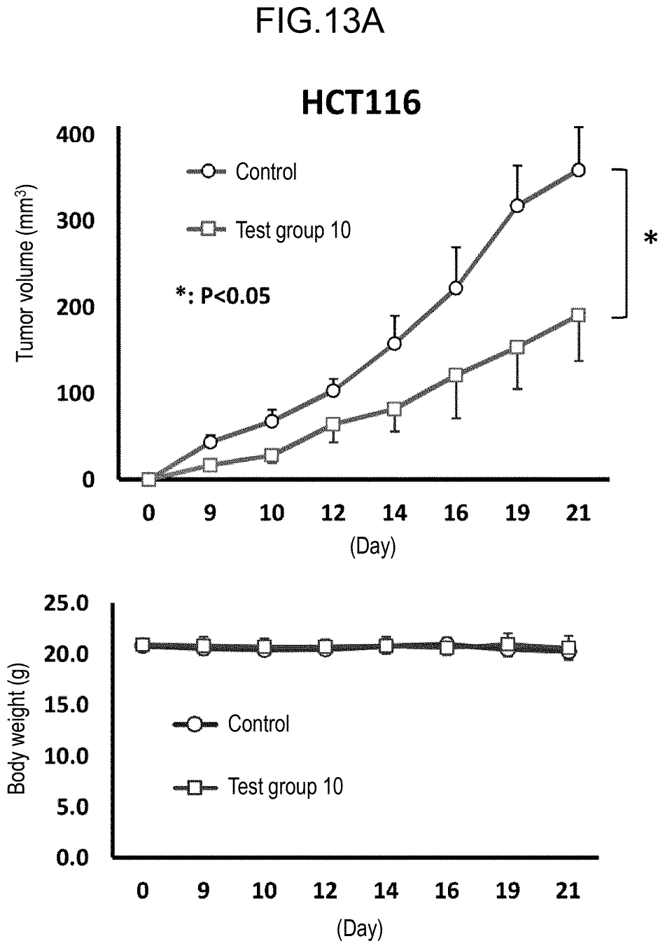

Furthermore, the apoptosis-inducing effects of the chelating agents of test groups 6 and 10 on tumors were confirmed. First, 100 μg/mL of the chelating agent of test group 6 or 50 μg/mL of the chelating agent of test group 10 was added to HSC-2 cells in a culture solution. After 48 hours of reaction, TUNEL Assay was carried out. Specifically, apoptosis induction was evaluated by the TUNEL staining method using a MK500 in situ Apoptosis Detection Kit (Takara Bio Inc.). As depicted in FIG. 11A, in the presence of the chelating agent of test group 6 and in the presence of the chelating agent of test group 10, green-stained cells indicating apoptosis were confirmed, revealing that these chelating agents had apoptosis-inducing effects on cancer cells.

Similarly, the apoptosis-inducing effects of the chelating agent of test group 6 or 10 on HCT116 cells were confirmed. 10 μg/mL of the chelating agent of test group 6 was added and 6.5 g/mL of the chelating agent of test group 10 was added to the cells. After 48 hours of reaction, TUNEL staining was carried out similarly to the above. As depicted in FIG. 11B, in the presence of the chelating agent of test group 6 and in the presence of the chelating agent of test group 10, green-stained cells indicating apoptosis were confirmed, revealing that these chelating agents had apoptosis-inducing effects on cancer cells.

Further, the apoptosis-inducing effects of the chelating agent of test group 6 or 10 on A549 cells were confirmed. 100 μg/mL of the chelating agent of test group 6 was added and 50 μg/mL of the chelating agent of test group 10 was added to the cells. After 48 hours of reaction, TUNEL staining was carried out similarly to the above. As depicted in FIG. 11C, in the presence of the chelating agent of test group 6 and in the presence of the chelating agent of test group 10, green-stained cells (FITC positive cells) indicating apoptosis were confirmed, revealing that these chelating agents had apoptosis-inducing effects on cancer cells.

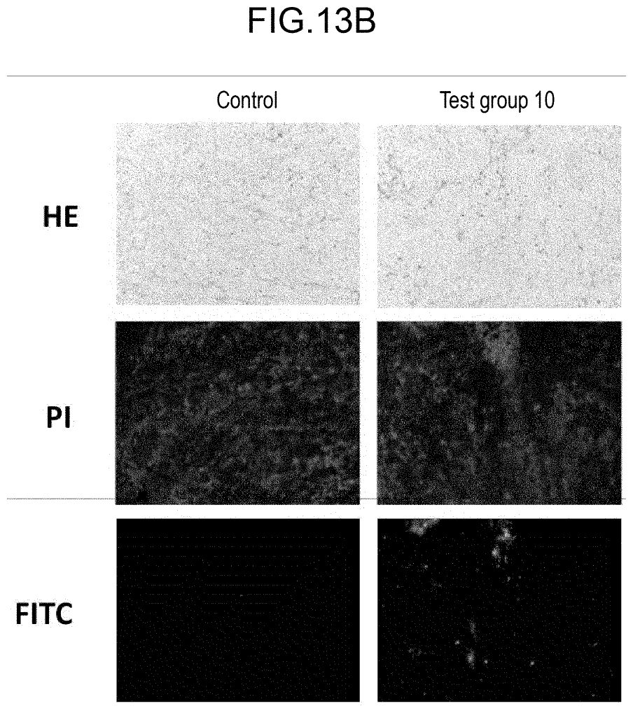

The cell lysate of each type of cells treated with the chelating agents of test groups 6 and 10 was Western-blotted, and then an increase or a decrease in apoptosis-related factors in treated cells was confirmed using the indicated antibodies. As a result, as depicted in FIG. 12, PARP cleaved fragments and caspase-3-cleaved fragments were confirmed in all of HSC-2 cells, MCF-7 cells, and A549 cells, when these cells were treated with either test group 6 or 10. Concentration-dependent apoptosis induction was also biochemically confirmed.

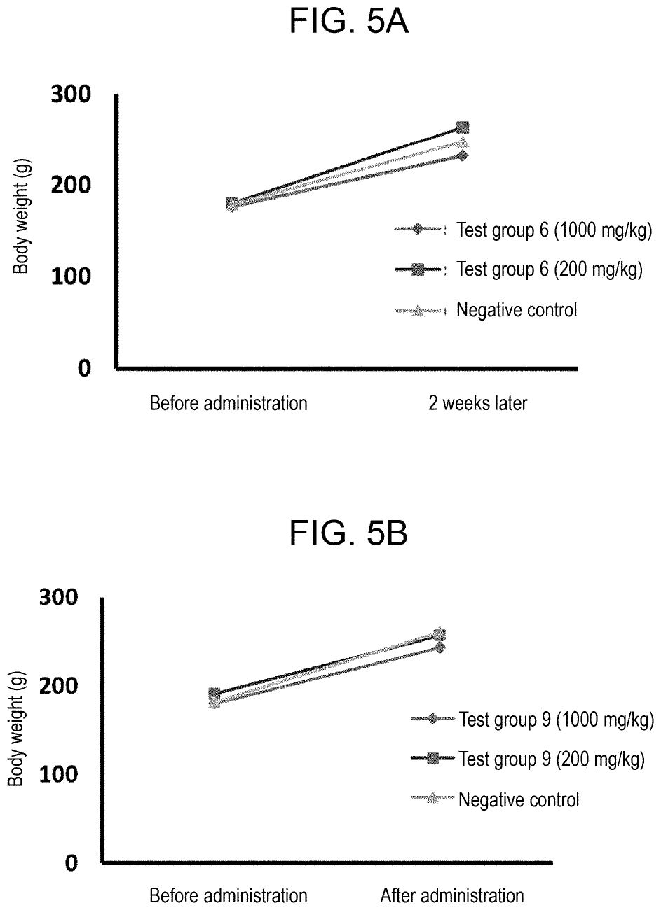

Antitumor effects were confirmed using animal models. Specifically, nude mice (BALB/c nu/nu, 6 weeks old: Clea Japan Inc.) were provided. Human lung cancer cell line (A549) was adjusted at 3×10⁶/mouse, and then mixed with Matrigel (BD Biosciences) at 1:1. The thus obtained mixture was administered to mice, thereby forming subcutaneous tumors. At 1 week after the formation of subcutaneous tumors, mice were divided into a negative control group (saline: 4 mice), a test group 9 administration group (test group 9: 3 mice), and a positive control group (deferasirox: 3 mice), and then subjected to oral administration. The dosage of deferasirox was 20 mg/kg/day (5 times/week administration) corresponding to that specified for humans in the package insert. Test group 9 was administered in the same dosage as that of deferasirox. Results were as depicted in FIGS. 4A and 4B.

As depicted in FIG. 4A, in the test group 9 administration group, antitumor effects were exhibited to an extent almost equivalent to that in the positive control group. Meanwhile, in the test group 9 administration group, no mice died during the experiment, and, as depicted in FIG. 4B, no significant change was observed in body weight.

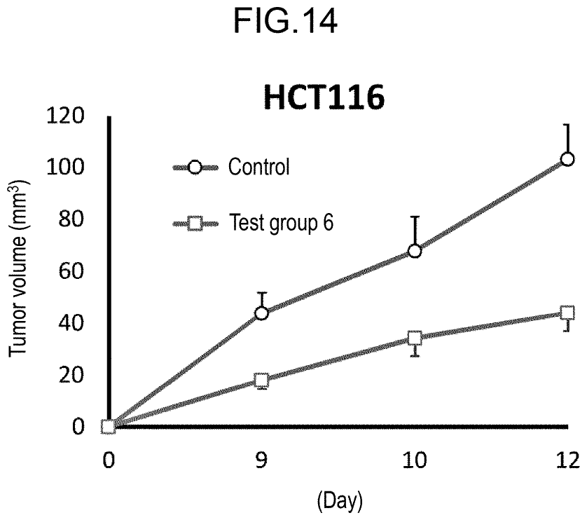

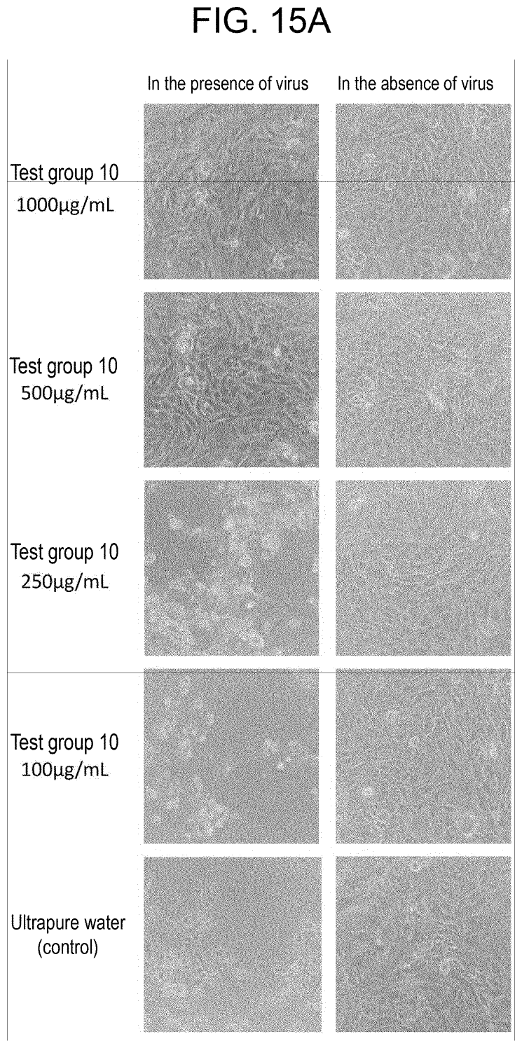

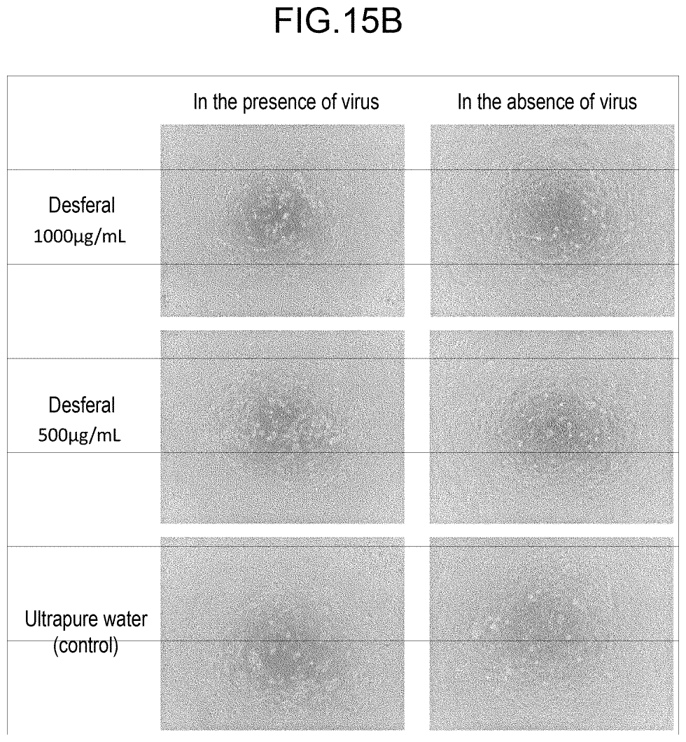

Next, the antitumor effects of the chelating agents of test groups 6 and 10 on HCT116-cell subcutaneous transplantation model mice, into which HCT116 cells had been subcutaneously transplanted, were confirmed. Mice prepared herein were nude mice (BALB/c nu/nu, 6 weeks old: Clea Japan Inc.). HCT116 cells were adjusted at 3×10⁶/mouse, and then mixed with Matrigel (BD Biosciences) at 1:1. The thus obtained mixture was administered to mice, thereby preparing subcutaneous tumors. The dosage was 200 mg/kg/day (5 times/week administration). As a result, as depicted in FIG. 13A, the chelating agent of test group 10 exhibited antitumor effects on subcutaneously-transplanted HCT116 cells, but no change in body weight was observed among the test group 10 administration group to which the chelating agent of test group 10 had been administered. Tissue sections of HCT116 cells subcutaneously transplanted into the test group 10 administration group were prepared, and then hematoxylin-eosin (HE) staining, and TUNEL staining were performed by the above-mentioned method. As a result, as depicted in FIG. 13B, FITC-derived green-stained cell images indicating apoptosis induction were confirmed in some of the tissues of the group to which the chelating agent of test group 10 had been administered. Similarly, as depicted in FIG. 14, the chelating agent of test group 6 exhibited antitumor effects on subcutaneously-transplanted HCT116 cells, and particularly the chelating agent of test group 6 exhibited significant antitumor effects on day 12 compared with that of the control. In addition, no body weight change due to administration was observed in the test group 6 administration group to which the chelating agent of test group 6 had been administered.

These results demonstrated that the chelating agents of the present invention exhibited antitumor effects on breast cancer such as breast cancer cells and epithelial cancer such as oral squamous cell carcinoma. Meanwhile, at the doses thereof exhibiting antitumor effects, no side effects were observed due to the administration of the antitumor agents, such as body weight loss.

According to the examples, the chelating agents of the present invention exhibited antitumor activity equivalent to that of existing iron chelating agents, but were observed to cause almost no side effects, revealing that the chelating agents of the present invention can be highly likely ideal anticancer agents.

### Example 3: Acute Toxicity Study

In this example, the chelating agents of the present invention were orally or intravenously administered to examine the toxicity.

### (1) Toxicity Study Through Peroral Administration

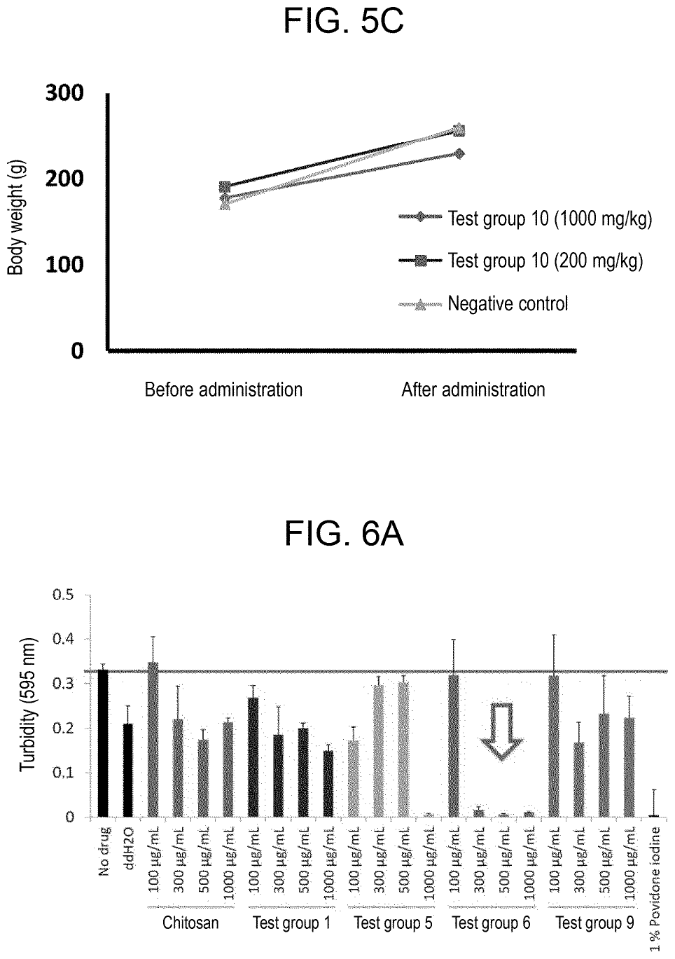

To each of 7-week-old JCL: SD rats divided into a test group 6 administration group (n=3), a test group 9 administration group (n=3), and a test group 10 administration group (n=3), the chelating agent of each test group was administered orally at a dose of 200 mg/kg body weight or 1000 mg/kg body weight. To a negative control group (saline administration; n=6), the same amount of saline was administered. On day 14 after administration, determining life or death, measurement of body weight, physical items (respiration, body temperature, behavior, etc.) were observed. Moreover, blood samples were taken from each group, and the right kidneys and the livers were removed. Results were as depicted in FIG. 5A to 5C and Tables 4 to 9.

As depicted in FIG. 5A to 5C, in all the rats to which the chelating agents of the test groups were administered, no significant effect on the growth was observed. Further, thin-layer sections were prepared from the kidneys and the livers, and then observed by hematoxylin-eosin staining. No histological abnormality was observed in all the groups to which any of the chelating agents of the test groups had been administered.

TABLE 4
Observation results of test group 6-receiving rats

| | | | At the start | | | | | |
|---|---|---|---|---|---|---|---|---|
| | | | Agent | | | At the end | | |
| Observation | | | Test group 6 | | | Negative control | | |
| methods | | Observations | Rat (1) | Rat (2) | Rat (3) | Rat (4) | Rat (5) | Rat (6) |
| Through the cage | | Appearance, | ○ | ○ | ○ | ○ | ○ | ○ |
| | | Hair coat | | | | | | |
| | | Consciousness, | ○ | ○ | ○ | ○ | ○ | ○ |
| | | Behavior | | | | | | |
| | | Convulsion, | ○ | ○ | ○ | ○ | ○ | ○ |
| | | etc. | | | | | | |
| | | Respiration | ○ | ○ | ○ | ○ | ○ | ○ |
| | | Posture | ○ | ○ | ○ | ○ | ○ | ○ |
| | | Acoustic | ○ | ○ | ○ | ○ | ○ | ○ |
| | | reflection | | | | | | |
| Handling | Holding | Lower | ○ | ○ | ○ | ○ | ○ | ○ |
| | the tail | abdomen | | | | | | |
| | Holding | Reactivity | ○ | ○ | ○ | ○ | ○ | ○ |
| | the body | Eyes, nose, | ○ | ○ | ○ | ○ | ○ | ○ |
| | | mouth | | | | | | |
| | | Skin | ○ | ○ | ○ | ○ | ○ | ○ |
| | | Chest and | ○ | ○ | ○ | ○ | ○ | ○ |
| | | abdomen | | | | | | |
| | | Muscle tone | ○ | ○ | ○ | ○ | ○ | ○ |
| | | Body | ○ | ○ | ○ | ○ | ○ | ○ |
| | | temperature | | | | | | |
| | | Respiratory | ○ | ○ | ○ | ○ | ○ | ○ |
| | | sound | | | | | | |

| | | | | | | | |
|---|---|---|---|---|---|---|---|
| On the cage | Behavior | ○ | ○ | ○ | ○ | ○ | ○ |
| | Head | ○ | ○ | ○ | ○ | ○ | ○ |

| | | | | | | |
|---|---|---|---|---|---|---|
| Body weight (g) | 174.48 | 178.68 | 180.37 | 183.87 | 166.14 | 179.98 |

*The dose for each of rat (1) and rat (2) is 1000 mg/kg, and the dose for rat (3) is 200 mg/kg.
*In this table, symbol ″○″ indicates that no abnormality was observed.

TABLE 5
Blood test results of test group 6-receivinq rats

| | Rat (1) | Rat (2) | Rat (3) | Unit |
|---|---|---|---|---|
| TP | 5.4 | 5.1 | 5.1 | g/dL |
| T-Bil | 0.01 | 0.05 | 0.03 | mg/dL |
| GLU | 159 | 199 | 212 | mg/dL |
| NEFA | 69 | 2 | 58 | mEQ/L |
| BUN | 22.2 | 28.9 | 27.2 | mg/dL |
| CRE | 0.31 | 0.48 | 0.37 | mg/dL |
| Na | 144 | 143 | 141 | mEQ/L |
| K | 4.5 | 4.1 | 3.6 | mEQ/L |
| Ca | 10.2 | 11.0 | 10.2 | mg/dL |
| AST | 179 | 52 | 58 | IU/L |
| ALT | 94 | 27 | 34 | IU/L |
| LD | 127 | 60 | 45 | IU/L |
| ALP | 437 | 471 | 745 | IU/L |
| γ-GTP | Less | Less | Less | IU/L |
| | than 3 | than 3 | than 3 | |

*The dose for each of rat (1) and rat (2) is 1000 mg/kg, and the dose for rat (3) is 200 mg/kg.

TABLE 6
Observation results of test group 9-receiving rats

| | | | At the start | | | | | |
|---|---|---|---|---|---|---|---|---|
| | | | Agent | | | At the end | | |
| Observation | | | Test group 9 | | | Negative control | | |
| methods | | Observations | Rat (1) | Rat (2) | Rat (3) | Rat (4) | Rat (5) | Rat (6) |
| Through the cage | | Appearance, | ○ | ○ | ○ | ○ | ○ | ○ |
| | | Hair coat | | | | | | |
| | | Consciousness, | ○ | ○ | ○ | ○ | ○ | ○ |
| | | Behavior | | | | | | |
| | | Convulsion, | ○ | ○ | ○ | ○ | ○ | ○ |
| | | etc. | | | | | | |
| | | Respiration | ○ | ○ | ○ | ○ | ○ | ○ |
| | | Posture | ○ | ○ | ○ | ○ | ○ | ○ |
| | | Acoustic | ○ | ○ | ○ | ○ | ○ | ○ |
| | | reflection | | | | | | |
| Handling | Holding | Lower | ○ | ○ | ○ | ○ | ○ | ○ |
| | the tail | abdomen | | | | | | |
| | Holding | Reactivity | ○ | ○ | ○ | ○ | ○ | ○ |
| | the body | Eyes, nose, | ○ | ○ | ○ | ○ | ○ | ○ |
| | | mouth | | | | | | |
| | | Skin | ○ | ○ | ○ | ○ | ○ | ○ |
| | | Chest and | ○ | ○ | ○ | ○ | ○ | ○ |
| | | abdomen | | | | | | |
| | | Muscle tone | ○ | ○ | ○ | ○ | ○ | ○ |
| | | Body | ○ | ○ | ○ | ○ | ○ | ○ |
| | | temperature | | | | | | |
| | | Respiratory | ○ | ○ | ○ | ○ | ○ | ○ |
| | | sound | | | | | | |

| | | | | | | | |
|---|---|---|---|---|---|---|---|
| On the cage | Behavior | ○ | ○ | ○ | ○ | ○ | ○ |
| | Head | ○ | ○ | ○ | ○ | ○ | ○ |

| | | | | | | |
|---|---|---|---|---|---|---|
| Body weight (g) | 177.11 | 183.15 | 190.97 | 181.53 | 177.45 | 186.27 |

*The dose for each of rat (1) and rat (2) is 1000 mg/kg, and the dose for rat (3) is 200 mg/kg.
*In this table, symbol ″○″ indicates that no abnormality was observed.

TABLE 7
Blood test results of test group 9-receivinq rats

| | Rat (1) | Rat (2) | Rat (3) | Unit |
|---|---|---|---|---|
| TP | 5.4 | 5.9 | 5.5 | g/dL |
| T-Bil | 0.06 | 0.06 | 0.05 | mg/dL |
| GLU | 195 | 301 | 214 | mg/dL |
| NEFA | 163 | 133 | 59 | mEQ/L |
| BUN | 22.5 | 24.4 | 22.8 | mg/dL |
| CRE | 0.36 | 0.36 | 0.37 | mg/dL |
| Na | 143 | 144 | 146 | mEQ/L |
| K | 3.6 | 4.8 | 4.7 | mEQ/L |
| Ca | 10.0 | 11.2 | 11.1 | mg/dL |
| AST | 59 | 58 | 54 | IU/L |
| ALT | 24 | 25 | 24 | IU/L |
| LD | 78 | 70 | 49 | IU/L |
| ALP | 563 | 502 | 695 | IU/L |
| γ-GTP | Less | Less | Less | IU/L |
| | than 3 | than 3 | than 3 | |

*The dose for each of rat (1) and rat (2) is 1000 mg/kg, and the dose for rat (3) is 200 mg/kg.

TABLE 8
Observation results of test group 10-receiving rats

| | | | At the start | | | | | |
|---|---|---|---|---|---|---|---|---|
| | | | Agent | | | At the end | | |
| Observation | | | Test group 10 | | | Negative control | | |
| methods | | Observations | Rat (1) | Rat (2) | Rat (3) | Rat (4) | Rat (5) | Rat (6) |
| Through the cage | | Appearance, | ○ | ○ | ○ | ○ | ○ | ○ |
| | | Hair coat | | | | | | |
| | | Consciousness, | ○ | ○ | ○ | ○ | ○ | ○ |
| | | Behavior | | | | | | |
| | | Convulsion, | ○ | ○ | ○ | ○ | ○ | ○ |
| | | etc. | | | | | | |
| | | Respiration | ○ | ○ | ○ | ○ | ○ | ○ |
| | | Posture | ○ | ○ | ○ | ○ | ○ | ○ |
| | | Acoustic | ○ | ○ | ○ | ○ | ○ | ○ |
| | | reflection | | | | | | |
| Handling | Holding | Lower | ○ | ○ | ○ | ○ | ○ | ○ |
| | the tail | abdomen | | | | | | |
| | Holding | Reactivity | ○ | ○ | ○ | ○ | ○ | ○ |
| | the body | Eyes, nose, | ○ | ○ | ○ | ○ | ○ | ○ |
| | | mouth | | | | | | |
| | | Skin | ○ | ○ | ○ | ○ | ○ | ○ |
| | | Chest and | ○ | ○ | ○ | ○ | ○ | ○ |
| | | abdomen | | | | | | |
| | | Muscle tone | ○ | ○ | ○ | ○ | ○ | ○ |
| | | Body | ○ | ○ | ○ | ○ | ○ | ○ |
| | | temperature | | | | | | |
| | | Respiratory | ○ | ○ | ○ | ○ | ○ | ○ |
| | | sound | | | | | | |

| | | | | | | | |
|---|---|---|---|---|---|---|---|
| On the cage | Behavior | ○ | ○ | ○ | ○ | ○ | ○ |
| | Head | ○ | ○ | ○ | ○ | ○ | ○ |

| | | | | | | |
|---|---|---|---|---|---|---|
| Body weight (g) | 170.73 | 185.29 | 170.83 | 181.53 | 177.45 | 186.27 |

*The dose for each of rat (1) and rat (2) is 1000 mg/kg, and the dose for rat (3) is 200 mg/kg.
*In this table, symbol ″○″ indicates that no abnormality was observed.

TABLE 9
Blood test results of test group 10-receiving rats

| | Rat (1) | Rat (2) | Rat (3) | Unit |
|---|---|---|---|---|
| TP | 5.4 | 5.7 | 5.5 | g/dL |
| T-Bil | 0.06 | 0.01 | 0.06 | mg/dL |
| GLU | 259 | 187 | 278 | mg/dL |
| NEFA | 18 | 77 | 111 | mEQ/L |
| BUN | 24.6 | 22.8 | 22.5 | mg/dL |
| CRE | 0.36 | 0.36 | 0.37 | mg/dL |
| Na | 142 | 143 | 146 | mEQ/L |
| K | 4.8 | 4.9 | 6.1 | mEQ/L |
| Ca | 10.6 | 10.5 | 11.2 | mg/dL |
| AST | 56 | 90 | 61 | IU/L |
| ALT | 25 | 25 | 33 | IU/L |
| LD | 57 | 562 | 53 | IU/L |
| ALP | 592 | 618 | 603 | IU/L |
| γ-GTP | Less | Less | Less | IU/L |
| | than 3 | than 3 | than 3 | |

*The dose for each of rat (1) and rat (2) is 1000 mg/kg, and the dose for rat (3) is 200 mg/kg.

As depicted in Tables 4 to 9 above, all the rats to which the chelating agents of the test groups were administered exhibited results almost the same as those of the negative control. This means that all the chelating agents of the test groups will not exhibit acute toxicity at detectable levels.

### (2) Toxicity Study Through Intravenous Administration

To each of 6-week-old Jcl: ICR mice divided into a test group 6 administration group (n=3), a test group 9 administration group (n=3), and a test group 10 administration group (n=3), the chelating agent of each test group was administered at a dose of 300 mg/kg body weight via tail vein. To a positive control group (administration; n=4), deferoxamine mesylate was administered at a dose of 300 mg/kg body weight via tail vein. On day 7 after administration, determining life or death, and physical items (respiration, body temperature, behavior, etc.) were observed.

As a result, all the mice of the control group to which deferoxamine mesylate was administered, died immediately after administration. However, 7 days of survival was confirmed for all the groups to which the chelating agents of the test groups were administered, and no significant change was apparently confirmed in regions other than the intravenous injection sites.

As described above, the chelating agents of the present invention were chelating agents selective for biologically unstable iron rather than for transferrin-bound iron required for living bodies, and exhibited almost no biological toxicity.

### Example 4: Evaluation of Antimicrobial Effects

In this example, antimicrobial effects on each starter were confirmed.

(1) S. aureus, P. aeruginosa and C. albicans

The dilution series of the chelating agent of each test group was prepared according to a standard method, microbes were inoculated, and then 24 hours later, the microbial growth was confirmed, thereby determining the minimum growth inhibition concentration of the chelating agent of each test group.

Microbes used herein were S. aureus (ATCC6538; Staphylococcus aureus), P. aeruginosa (ATCC9027; Pseudomonas aeruginosa) and C. albicans (ATCC10231; Candida albicans).

Drugs used herein were oxacillin, vancomycin and deferasirox as positive controls.

Results were as depicted in Table 10.

TABLE 10

| | MIC (μg/mL) | | |
|---|---|---|---|
| | S. | P. | C. |
| | aureus | aeruginosa | albicans |
| Oxacillin | 0.25 | 1024 | |
| Vancomycin | 1.6 | >50 | |
| Deferasirox | 250 | >250 | >250 |
| Test group 1 | 50 | >800 | >800 |
| Test group 2 | 200 | >800 | 12.5-100 |
| Test group 4 | 200-400 | >800 | >800 |
| Test group 5 | 800 | >800 | 800 |
| Test group 6 | 400 | 400 | >800 |
| Test group 7 | >800 | >800 | >800 |
| Test group 8 | 800 | >800 | >800 |
| Test group 9 | >500 | >500 | >500 |

As depicted in Table 10, test groups 1, 2, 4 to 6 and 8 were revealed to have antimicrobial effects on S. aureus. Of these groups, test groups 1, 2, 4 and 6 exhibited strong antimicrobial effects on S. aureus.

Further, test group 6 exhibited antimicrobial effects on P. aeruginosa.

Further, particularly test groups 2 and 5 exhibited antimicrobial effects on C. albicans, and of these groups, test group 2 exhibited particularly strong antimicrobial effects on C. albicans.

Furthermore, antimicrobial effects on microbes in the oral region were confirmed.

(2) Streptococcus mutans

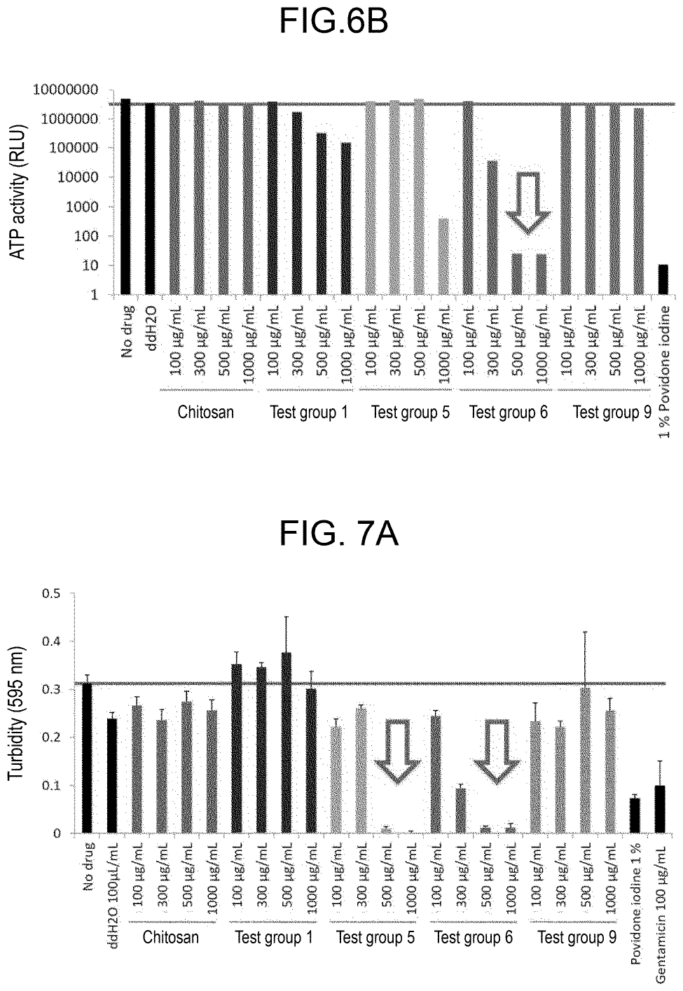

First, the antibacterial activity of a dental caries-causing bacterium, S. mutans (ATCC25175) was examined. With the use of a liquid medium prepared by adding a yeast extract and the chelating agent of each test group to TSB medium, aerobic culture was performed at 37° C. for 18 hours, and then turbidity was found from optical density (OD570 or 595 nm). Cell activity was measured using an ATP assay kit (Kikkoman Corporation). Results were as depicted in FIGS. 6A and 6B.

As depicted in FIGS. 6A and B, the chelating agents of test groups 1, 5 and 6 exhibited strong antimicrobial effects on the above dental caries-causing bacterium, S. mutans, and particularly test group 6 exhibited very strong antibacterial activity against the bacterium.

(3) Aggregatibacter actinomycetemcomitans

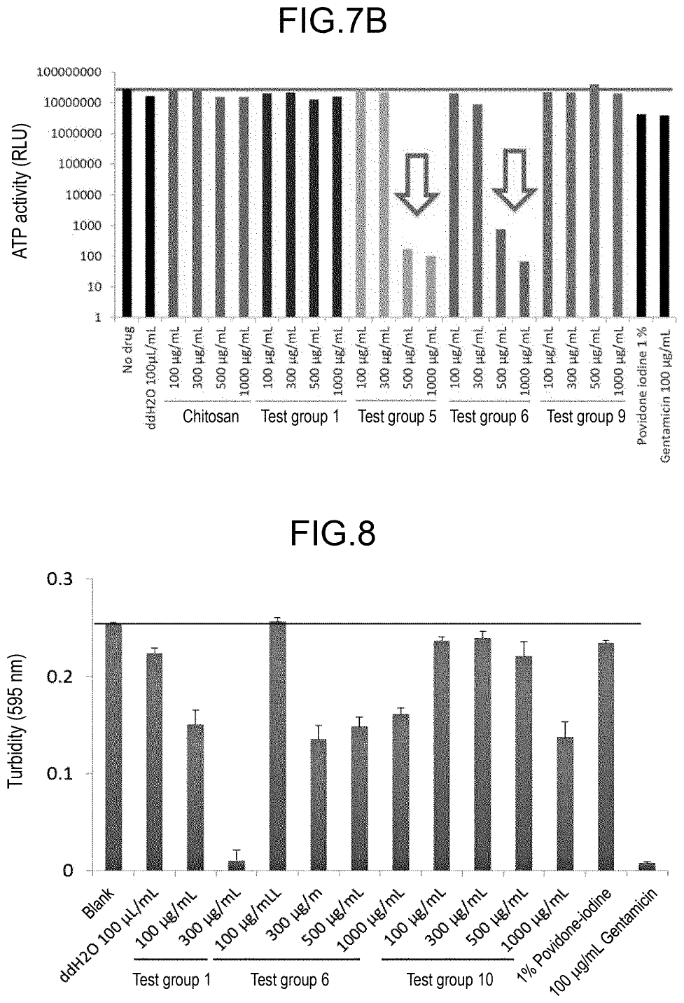

A bacterial strain of one of periodontopathic bacteria, A. actinomycetemcomitans, used herein was AaY4 (ATCC). With the use of a liquid medium prepared by adding a yeast extract, sodium bicarbonate and the chelating agent of each test group to TSB medium, anaerobic culture (Anaeropack-anaerobic) was performed at 37° C. for 18 hours. Turbidity was measured using absorbance (OD570 or 595 nm), and cell activity was measured using an ATP assay kit. Results were as depicted in FIGS. 7A and 7B.

As depicted in FIGS. 7A and 7B, the chelating agent of test groups 5 and 6 exhibited strong antimicrobial effects on A. actinomycetemcomitans.

(4) Porphyromonas gingivalis

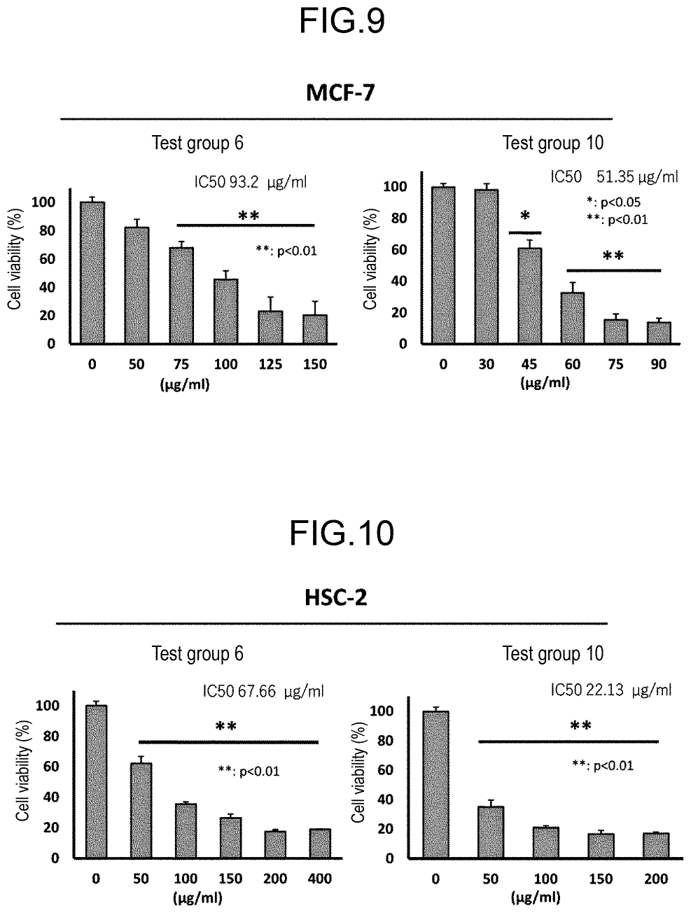

A bacterial strain of one of periodontopathic bacteria, gram-negative bacillus, P. gingivalis, used herein was Pg W83. With the use of a liquid medium prepared by adding the chelating agent of each test group to a modified GAM bouillon medium, anaerobic culture (Anaeropack.anaerobic) was performed at 37° C. for 18 hours. Turbidity was found from optical density (OD595 nm). Results were as depicted in FIG. 8.

As depicted in FIG. 8, all test groups 1, 6 and 10 exhibited antimicrobial effects on P. gingivalis. In addition, when cell activity was measured for test group 1 using an ATP assay kit in a manner similar to the above, a significant decrease in cell activity could be confirmed.

As described above, the chelating agents of the present invention are chelating agents having selectivity for biologically unstable iron rather than for transferrin-bound iron, and chelate iron ions not required for living bodies. However, the chelating agents of the present invention were observed to have almost no biological toxicity, but were observed to have anticancer effects and antimicrobial effects.

### Example 5: Verification of Antiviral Effects

In this example, if the chelating agents of the present invention had antiviral effects was examined.

MDCK cells (canine renal tubule epithelial cells) were infected with a human influenza virus, and then an effect of suppressing the viral infection of MDCK cells was confirmed. Specifically, PR8 [A/PUERTORICO/8/34 (H1N1)] was adjusted at TCID₅₀=2.81×10⁶/mL as the influenza virus, the viral liquid and an equivalent amount of the chelating agent of test group 10 (concentration: 100 μg/mL, 250 μg/mL, 500 μg/mL, or 1000 μg/mL) were mixed and then left to stand at room temperature for 30 minutes. The above-prepared viral liquid was added to MDCK cells. After 48 hours, cells were observed, living cells (specifically, non-infected cells) remaining on the bottom of the culture dish were observed under a microscope. Results were as depicted in FIG. 16A. As depicted in FIG. 16A, the chelating agent of test group 10 was observed to have the effect of suppressing viral infection at a concentration of 500 μg/mL. On the other hand, similarly when specifically PR8 [A/PUERTORICO/8/34 (H1N1)] was adjusted at TCID₅₀=2.81×10⁶/mL as an influenza virus, and then the viral liquid and an equivalent amount of Desferal (concentration: 500 μg/mL, or 1000 μg/mL) were mixed, the effect of suppressing viral infection was not observed as depicted in FIG. 16B.

Accordingly, it was revealed that the chelating agents of the present invention have antiviral effects and particularly have effects of protecting cells from viral infection. Hence, the chelating agents of the present invention can be used for prophylaxis (control) and treatment of viral infection. The chelating agents of the present invention do not chelate transferrin-bound iron, and are selective for biologically unstable iron, so that the agents are believed to have only few side effects, and be useful as antiviral agents.

1: A pharmaceutical composition, comprising an iron chelating agent, wherein
the iron chelating agent has a substrate selected from the group consisting of a polymer backbone, glucosamine, and histidine; and an aromatic ring bonded to the substrate through an —NH—CH₂— bond, wherein the aromatic ring has one or two first functional groups, which are each a hydroxyl group; and one or two second functional groups selected from the group consisting of a hydroxyl group, a carboxylic acid group, and a functional group of formula (I): 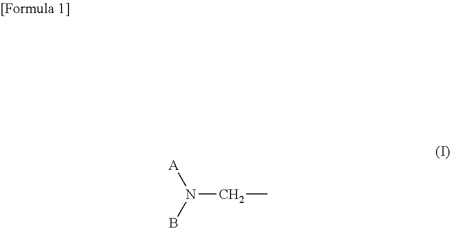
wherein A is —CH₃, —CH₂—CH₃, —CH₂—C₆H₅, —CH₂—C₅H₄N, or —CH₂—COOH; and B is —CH₂—COOH, and wherein
each of the second functional groups is located in the ortho position relative to at least one of the first functional groups.

2: The pharmaceutical composition according to claim 1, wherein the polymer backbone is a chitosan backbone.

3: The pharmaceutical composition according to claim 1, wherein the aromatic ring has the following structure: 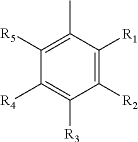
wherein any one of R₁ to R₅ is OH; the ring has at least one group of OH or COOH in the ortho position relative to the OH; and the other groups are selected from the group consisting of H, OH, COOH, CH₃, and —N(CH₃)—CH₂—COOH.

4: The pharmaceutical composition according to claim 1, wherein the substrate is glucosamine.

5: The pharmaceutical composition according to claim 4, wherein the aromatic ring has the following structure: 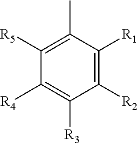
wherein any one of R₁ to R₅ is OH; the ring has at least one OH or COOH in the ortho position relative to the OH; and the other groups are selected from the group consisting of H, OH, COOH, CH₃, and —N(CH₃)—CH₂—COOH.

6: The pharmaceutical composition according to claim 5, wherein R₁ is H or OH; R₂ and R₃ are each OH; and R₄ and R₅ are each H.

7: The pharmaceutical composition according to claim 1, wherein the iron chelating agent has the following structure: 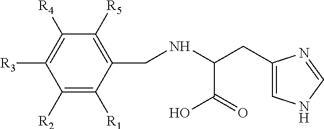
wherein any one of R₁ to R₅ is OH; the ring has at least one OH or COOH in the ortho position relative to the OH; and the other groups are selected from the group consisting of H, OH, COOH, CH₃, and —N(CH₃)—CH₂—COOH.

8: The pharmaceutical composition according to claim 7, wherein the chelating agent is in the form of hydrochloride salt.

9: The pharmaceutical composition according to claim 7, wherein any one of R₁ to R₅ is OH; and the ring has at least OH and COOH in the ortho position relative to the OH.

10: The pharmaceutical composition according to claim 9, wherein
R₁ to R₃ are each OH; and R₄ and R₅ are each H; or
R₁ is H; one of R₂ and R₃ is OH; and the other is COOH; and R₄ and R₅ are H.

11: An antimicrobial agent, comprising an iron chelating agent, wherein
the iron chelating agent has a substrate selected from the group consisting of a polymer backbone, glucosamine, and histidine; and an aromatic ring bonded to the substrate through an —NH—CH₂— bond, wherein the aromatic ring has one or two first functional groups, which are each a hydroxyl group; and one or two second functional groups selected from the group consisting of a hydroxyl group, a carboxylic acid group, and a functional group of formula (I): 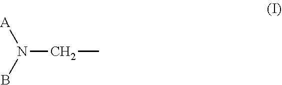
wherein A is —CH₃, —CH₂—CH₃, —CH₂—C₆H₅, —CH₂—C₅H₄N, or —CH₂—COOH; and B is —CH₂—COOH, and wherein
the second functional group is located in the ortho position relative to at least one of the first functional groups.

12: The antimicrobial agent according to claim 11, wherein the substrate is glucosamine.

13: The antimicrobial agent according to claim 11, wherein
the iron chelating agent has glucosamine; and an aromatic ring bonded to glucosamine through an —NH—CH₂— bond, wherein the aromatic ring has one or two first functional groups, which are each a hydroxyl group; and one or two second functional groups selected from the group consisting of a hydroxyl group, a carboxylic acid group, and a functional group of formula (I): 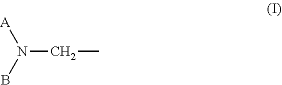
wherein A is —CH₃, —CH₂—CH₃, —CH₂—C₆H₅, —CH₂—C₅H₄N, or —CH₂—COOH; and B is —CH₂—COOH, and wherein
the second functional group is located in the ortho position relative to at least one of the first functional groups.

14: The antimicrobial agent according to claim 12, wherein the aromatic ring has the following structure: 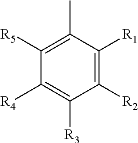
wherein any one of R₁ to R₅ is OH; the ring at least has OH or COOH in the ortho position relative to the OH; and the other groups are selected from the group consisting of H, OH, COOH, CH₃, and —N(CH₃)—CH₂—COOH.

15: The antimicrobial agent according to claim 14, wherein R₁ is H or OH; R₂ and R₃ are each OH; and R₄ and R₅ are each H.

16: The antimicrobial agent according to claim 11, wherein
the antimicrobial agent is an antimicrobial agent for use against an oral bacterium selected from the group consisting of S. mutans, A. actinomycetemcomitans and P. gingivalis.

17: The antimicrobial agent according to claim 15, wherein
the antimicrobial agent is an antimicrobial agent for use against an oral bacterium selected from the group consisting of S. mutans, A. actinomycetemcomitans and P. gingivalis.

18: The antimicrobial agent according to claim 11, wherein
the antimicrobial agent is an antimicrobial agent for use against an oral bacterium selected from the group consisting of S. mutans and P. gingivalis, and
the iron chelating agent has chitosan as a substrate.

19: The antimicrobial agent according to claim 11, wherein
the antimicrobial agent is an antimicrobial agent for use against S. aureus or C. albicans, and
the iron chelating agent has a chitosan backbone as the polymer backbone.

20: The antimicrobial agent according to claim 15, wherein
the antimicrobial agent is an antimicrobial agent for use against S. aureus.

21: The antimicrobial agent according to claim 15, wherein
R₁ to R₃ are each OH; and R₄ and R₅ are each H, and
the antimicrobial agent is an antimicrobial agent for use against P. aeruginosa.

22: A pharmaceutical composition, comprising an iron chelating agent having selectivity for biologically unstable iron rather than for transferrin-bound iron.

23: An antimicrobial agent, comprising an iron chelating agent having selectivity for biologically unstable iron rather than for transferrin-bound iron.

24: A pharmaceutical composition, comprising an iron chelating agent, wherein
the iron chelating agent has a substrate selected from the group consisting of a polymer backbone, glucosamine, and histidine; and an aromatic ring, wherein the aromatic ring has one or two first functional groups, which are each a hydroxyl group; and one or two second functional groups selected from the group consisting of a hydroxyl group, a carboxylic acid group, and a functional group of formula (I): 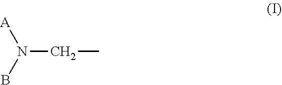
wherein A is —CH₃, —CH₂—CH₃, —CH₂—C₆H₅, —CH₂—C₅H₄N, or —CH₂—COOH; and B is —CH₂—COOH, and wherein
the second functional group is located in the ortho position relative to at least one of the first functional groups.

25: The pharmaceutical composition according to claim 24, wherein the aromatic ring has the following structure: 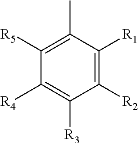
wherein any one of R₁ to R₅ is OH; the ring has at least OH or COOH in the ortho position relative to the OH; and the other groups are selected from the group consisting of H, OH, COOH, CH₃, and —N(CH₃)—CH₂—COOH.

26: The pharmaceutical composition according to claim 25, wherein R₁ is H or OH; R₂ and R₃ are each OH; and R₄ and R₅ are each H.

27: The pharmaceutical composition according to claim 25, wherein R₁ and R₅ are each H; and R₂ to R₄ are each OH.

28. (canceled)
